SEQUENCE LISTING (27 sequences)

SEQ ID NO: 1 - DNA, 2478 bp, Homo sapiens
  human ST3 beta-galactoside alpha-2,3-sialyltransferase 3 (ST3Gal-III) cDNA, transcript variant 1
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccagtaagtatagtcactctagctcaccccaggagaagcctgttgcagattcagtggttctttcctttgactccgctggacaaacactaggctcagagtatgatcggttgggcttcctcctgaatctggactctaaactctcaccgaggactctctgcacggtggtttttggccttgactgcatattggaatcacctggagagcctaaaaaattactgatgcctgcatcccaccctctagagattttgaagtcactgagcgaggacgcagcctttgcattaggatttttaaagctgcccaggcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagacaatctgatcaaagccatcttgtcagtcaccaaagagtaccgcctgacccctgccttggacagcctccgctgccgccgctgcatcatcgtgggcaatggaggcgttcttgccaacaagtctctggggtcacgaattgacgactatgacattgtggtgagactgaattcagcaccagtgaaaggctttgagaaggacgtgggcagcaaaacgacactgcgcatcacctaccccgagggcgccatgcagcggcctgagcagtacgagcgcgattctctctttgtcctcgccggcttcaagtggcaggactttaagtggttgaaatacatcgtctacaaggagagagtgagtgcatcggatggcttctggaaatctgtggccactcgagtgcccaaggagccccctgagattcgaatcctcaacccatatttcatccaggaggccgccttcaccctcattggcctgcccttcaacaatggcctcatgggccgggggaacatccctacccttggcagtgtggcagtgaccatggcactacacggctgtgacgaggtggcagtcgcaggatttggctatgacatgagcacacccaacgcacccctgcactactatgagaccgttcgcatggcagccatcaaagagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 2 - protein, 444 aa, Homo sapiens
  human ST3 beta-galactoside alpha-2,3-sialyltransferase 3 (ST3Gal-III), isoform a, isoform A8
MGLLVFVRNLLLALCLFLVLGFLYYSAWKLHLLQWEEDSSKYSHSSSPQEKPVADSVVLSFDSAGQTLGSEYDRLGFLLNLDSKLSPRTLCTVVFGLDCILESPGEPKKLLMPASHPLEILKSLSEDAAFALGFLKLPRPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKESWTHNIQREKEFLRKLVKARVITDLSSGI

SEQ ID NO: 3 - DNA, 2316 bp, Homo sapiens
  human ST3 beta-galactoside alpha-2,3-sialyltransferase 3 (ST3Gal-III) cDNA, transcript variant 2
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccagtaagtatagtcactctagctcaccccaggagaagcctgttgcagattcagtggttctttcctttgactccgctggacaaacactaggctcagagtatgatcggttgggcttcctcctgaatctggactctaaactgcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagacaatctgatcaaagccatcttgtcagtcaccaaagagtaccgcctgacccctgccttggacagcctccgctgccgccgctgcatcatcgtgggcaatggaggcgttcttgccaacaagtctctggggtcacgaattgacgactatgacattgtggtgagactgaattcagcaccagtgaaaggctttgagaaggacgtgggcagcaaaacgacactgcgcatcacctaccccgagggcgccatgcagcggcctgagcagtacgagcgcgattctctctttgtcctcgccggcttcaagtggcaggactttaagtggttgaaatacatcgtctacaaggagagagtgagtgcatcggatggcttctggaaatctgtggccactcgagtgcccaaggagccccctgagattcgaatcctcaacccatatttcatccaggaggccgccttcaccctcattggcctgcccttcaacaatggcctcatgggccgggggaacatccctacccttggcagtgtggcagtgaccatggcactacacggctgtgacgaggtggcagtcgcaggatttggctatgacatgagcacacccaacgcacccctgcactactatgagaccgttcgcatggcagccatcaaagagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 4 - protein, 390 aa, Homo sapiens
  human ST3 beta-galactoside alpha-2,3-sialyltransferase 3 (ST3Gal-III), isoform b, isoform A1
MGLLVFVRNLLLALCLFLVLGFLYYSAWKLHLLQWEEDSSKYSHSSSPQEKPVADSVVLSFDSAGQTLGSEYDRLGFLLNLDSKLPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKESWTHNIQREKEFLRKLVKARVITDLSSGI

SEQ ID NO: 5 - DNA, 1675 bp, Homo sapiens
  human ST3 beta-galactoside alpha-2,3-sialyltransferase 3 (ST3Gal-III) cDNA, transcript variant 3
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccagtaagtatagtcactctagctcaccccaggagaagcctgttgcagattcagtggttctttcctttgactccgctggacaaacactaggctcagagtatgatcggttgggcttcctcctgaatctggactctaaactgcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 6 - protein, 170 aa, Homo sapiens
  human ST3 beta-galactoside alpha-2,3-sialyltransferase 3 (ST3Gal-III), isoform c, isoform A7
MGLLVFVRNLLLALCLFLVLGFLYYSAWKLHLLQWEEDSSKYSHSSSPQEKPVADSVVLSFDSAGQTLGSEYDRLGFLLNLDSKLPAELATKYANFSEGACKPGYASALMTAIFPRFSKPAPMFLDDSFRKWARIREFVPPFGIKGQVLDAQYPARERVSAEAGESSRHH

SEQ ID NO: 7 - DNA, 1977 bp, Homo sapiens
  human ST3 beta-galactoside alpha-2,3-sialyltransferase 3 (ST3Gal-III) cDNA, transcript variant 4
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccaattcagtggttctttcctttgactccgctggacaaacactaggctcagagtatgatcggttgggcttcctcctgaatctggactctaaactgcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagacaatctgatcaaagccatcttgtcagtcaccaaagagtaccgcctgacccctgccttggacagcctccgctgccgccgctgcatcatcgtgggcaatggaggcgttcttgccaacaagtctctggggtcacgaattgacgactatgacattgtggtgagactgaattcagcaccagtgaaaggctttgagaaggacgtgggcagcaaaacgacactgcgcatcacctaccccgagggcgccatgcagcggcctgagcagtacgagcgcgattctctctttgtcctcgccggcttcaagtggcaggactttaagtggttgaaatacatcgtctacaaggagagagtgtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 8 - protein, 277 aa, Homo sapiens
  human ST3 beta-galactoside alpha-2,3-sialyltransferase 3 (ST3Gal-III), isoform d, isoform B4
MGLLVFVRNLLLALCLFLVLGFLYYSAWKLHLLQWEEDSNSVVLSFDSAGQTLGSEYDRLGFLLNLDSKLPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSWTHNIQREKEFLRKLVKARVITDLSSGI

SEQ ID NO: 9 - DNA, 1630 bp, Homo sapiens
  human ST3 beta-galactoside alpha-2,3-sialyltransferase 3 (ST3Gal-III) cDNA, transcript variant 5
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccaattcagtggttctttcctttgactccgctggacaaacactaggctcagagtatgatcggttgggcttcctcctgaatctggactctaaactgcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 10 - protein, 155 aa, Homo sapiens
  human ST3 beta-galactoside alpha-2,3-sialyltransferase 3 (ST3Gal-III), isoform e, isoform B7
MGLLVFVRNLLLALCLFLVLGFLYYSAWKLHLLQWEEDSNSVVLSFDSAGQTLGSEYDRLGFLLNLDSKLPAELATKYANFSEGACKPGYASALMTAIFPRFSKPAPMFLDDSFRKWARIREFVPPFGIKGQVLDAQYPARERVSAEAGESSRHH

SEQ ID NO: 11 - DNA, 2433 bp, Homo sapiens
  human ST3 beta-galactoside alpha-2,3-sialyltransferase 3 (ST3Gal-III) cDNA, transcript variant 6
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccaattcagtggttctttcctttgactccgctggacaaacactaggctcagagtatgatcggttgggcttcctcctgaatctggactctaaactctcaccgaggactctctgcacggtggtttttggccttgactgcatattggaatcacctggagagcctaaaaaattactgatgcctgcatcccaccctctagagattttgaagtcactgagcgaggacgcagcctttgcattaggatttttaaagctgcccaggcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagacaatctgatcaaagccatcttgtcagtcaccaaagagtaccgcctgacccctgccttggacagcctccgctgccgccgctgcatcatcgtgggcaatggaggcgttcttgccaacaagtctctggggtcacgaattgacgactatgacattgtggtgagactgaattcagcaccagtgaaaggctttgagaaggacgtgggcagcaaaacgacactgcgcatcacctaccccgagggcgccatgcagcggcctgagcagtacgagcgcgattctctctttgtcctcgccggcttcaagtggcaggactttaagtggttgaaatacatcgtctacaaggagagagtgagtgcatcggatggcttctggaaatctgtggccactcgagtgcccaaggagccccctgagattcgaatcctcaacccatatttcatccaggaggccgccttcaccctcattggcctgcccttcaacaatggcctcatgggccgggggaacatccctacccttggcagtgtggcagtgaccatggcactacacggctgtgacgaggtggcagtcgcaggatttggctatgacatgagcacacccaacgcacccctgcactactatgagaccgttcgcatggcagccatcaaagagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 12 - protein, 429 aa, Homo sapiens
  human ST3 beta-galactoside alpha-2,3-sialyltransferase 3 (ST3Gal-III), isoform f, isoform B8
MGLLVFVRNLLLALCLFLVLGFLYYSAWKLHLLQWEEDSNSVVLSFDSAGQTLGSEYDRLGFLLNLDSKLSPRTLCTVVFGLDCILESPGEPKKLLMPASHPLEILKSLSEDAAFALGFLKLPRPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKESWTHNIQREKEFLRKLVKARVITDLSSGI

SEQ ID NO: 13 - DNA, 2223 bp, Homo sapiens
  human ST3 beta-galactoside alpha-2,3-sialyltransferase 3 (ST3Gal-III) cDNA, transcript variant 7
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccaagtatgatcggttgggcttcctcctgaatctggactctaaactgcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagacaatctgatcaaagccatcttgtcagtcaccaaagagtaccgcctgacccctgccttggacagcctccgctgccgccgctgcatcatcgtgggcaatggaggcgttcttgccaacaagtctctggggtcacgaattgacgactatgacattgtggtgagactgaattcagcaccagtgaaaggctttgagaaggacgtgggcagcaaaacgacactgcgcatcacctaccccgagggcgccatgcagcggcctgagcagtacgagcgcgattctctctttgtcctcgccggcttcaagtggcaggactttaagtggttgaaatacatcgtctacaaggagagagtgagtgcatcggatggcttctggaaatctgtggccactcgagtgcccaaggagccccctgagattcgaatcctcaacccatatttcatccaggaggccgccttcaccctcattggcctgcccttcaacaatggcctcatgggccgggggaacatccctacccttggcagtgtggcagtgaccatggcactacacggctgtgacgaggtggcagtcgcaggatttggctatgacatgagcacacccaacgcacccctgcactactatgagaccgttcgcatggcagccatcaaagagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 14 - protein, 359 aa, Homo sapiens
  human ST3 beta-galactoside alpha-2,3-sialyltransferase 3 (ST3Gal-III), isoform g, isoform C1
MGLLVFVRNLLLALCLFLVLGFLYYSAWKLHLLQWEEDSKYDRLGFLLNLDSKLPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKESWTHNIQREKEFLRKLVKARVITDLSSGI

SEQ ID NO: 15 - DNA, 1582 bp, Homo sapiens
  human ST3 beta-galactoside alpha-2,3-sialyltransferase 3 (ST3Gal-III) cDNA, transcript variant 8
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccaagtatgatcggttgggcttcctcctgaatctggactctaaactgcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 16 - protein, 139 aa, Homo sapiens
  human ST3 beta-galactoside alpha-2,3-sialyltransferase 3 (ST3Gal-III), isoform h, isoform C7
MGLLVFVRNLLLALCLFLVLGFLYYSAWKLHLLQWEEDSKYDRLGFLLNLDSKLPAELATKYANFSEGACKPGYASALMTAIFPRFSKPAPMFLDDSFRKWARIREFVPPFGIKGQVLDAQYPARERVSAEAGESSRHH

SEQ ID NO: 17 - DNA, 2385 bp, Homo sapiens
  human ST3 beta-galactoside alpha-2,3-sialyltransferase 3 (ST3Gal-III) cDNA, transcript variant 9
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccaagtatgatcggttgggcttcctcctgaatctggactctaaactctcaccgaggactctctgcacggtggtttttggccttgactgcatattggaatcacctggagagcctaaaaaattactgatgcctgcatcccaccctctagagattttgaagtcactgagcgaggacgcagcctttgcattaggatttttaaagctgcccaggcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagacaatctgatcaaagccatcttgtcagtcaccaaagagtaccgcctgacccctgccttggacagcctccgctgccgccgctgcatcatcgtgggcaatggaggcgttcttgccaacaagtctctggggtcacgaattgacgactatgacattgtggtgagactgaattcagcaccagtgaaaggctttgagaaggacgtgggcagcaaaacgacactgcgcatcacctaccccgagggcgccatgcagcggcctgagcagtacgagcgcgattctctctttgtcctcgccggcttcaagtggcaggactttaagtggttgaaatacatcgtctacaaggagagagtgagtgcatcggatggcttctggaaatctgtggccactcgagtgcccaaggagccccctgagattcgaatcctcaacccatatttcatccaggaggccgccttcaccctcattggcctgcccttcaacaatggcctcatgggccgggggaacatccctacccttggcagtgtggcagtgaccatggcactacacggctgtgacgaggtggcagtcgcaggatttggctatgacatgagcacacccaacgcacccctgcactactatgagaccgttcgcatggcagccatcaaagagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 18 - protein, 413 aa, Homo sapiens
  human ST3 beta-galactoside alpha-2,3-sialyltransferase 3 (ST3Gal-III), isoform i, isoform C8
MGLLVFVRNLLLALCLFLVLGFLYYSAWKLHLLQWEEDSKYDRLGFLLNLDSKLSPRTLCTVVFGLDCILESPGEPKKLLMPASHPLEILKSLSEDAAFALGFLKLPRPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRLNSAPVKGFEKDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMSTPNAPLHYYETVRMAAIKESWTHNIQREKEFLRKLVKARVITDLSSGI

SEQ ID NO: 19 - DNA, 2303 bp, Homo sapiens
  human ST3 beta-galactoside alpha-2,3-sialyltransferase 3 (ST3Gal-III) cDNA, transcript variant 11
actatggcggcgcccatgcagcccagcgcgttgtgggctcccgccggggtcccccgcggctgtcgccgccgcctacgccgctgcctccgccttcctgccccgcgtcgggccgggcgccacctcccccctgcctccctctccgctgtggtcatttaggaaatcgtaaatcatgtgaagatgggactcttggtatttgtgcgcaatctgctgctagccctctgcctctttctggtactgggatttttgtattattctgcgtggaagctacacttactccagtgggaggaggactccaattcagtggttctttcctttgactccgctggacaaacactaggctcagagtatgatcggttgggcttcctcctgaatctggactctaaactgcctgctgaattagccaccaagtacgcaaacttttcagagggagcttgcaagcctggctatgcttcagccttgatgacggccatcttcccccggttctccaagccagcacccatgttcctggatgactcctttcgcaagtgggctagaatccgggagttcgtgccgccttttgggatcaaaggtcaagacaatctgatcaaagccatcttgtcagtcaccaaagagtaccgcctgacccctgccttggacagcctccgctgccgccgctgcatcatcgtgggcaatggaggcgttcttgccaacaagtctctggggtcacgaattgacgactatgacattgtggtgaggcccaggctctgagtgggcctgctctctgtagactgaattcagcaccagtgaaaggctttgagaaggacgtgggcagcaaaacgacactgcgcatcacctaccccgagggcgccatgcagcggcctgagcagtacgagcgcgattctctctttgtcctcgccggcttcaagtggcaggactttaagtggttgaaatacatcgtctacaaggagagagtgagtgcatcggatggcttctggaaatctgtggccactcgagtgcccaaggagccccctgagattcgaatcctcaacccatatttcatccaggaggccgccttcaccctcattggcctgcccttcaacaatggcctcatgggccgggggaacatccctacccttggcagtgtggcagtgaccatggcactacacggctgtgacgaggtggcagtcgcaggatttggctatgacatgagcacacccaacgcacccctgcactactatgagaccgttcgcatggcagccatcaaagagtcctggacgcacaatatccagcgagagaaagagtttctgcggaagctggtgaaagctcgcgtcatcactgatctaagcagtggcatctgagtgggcccagcacatggccatagaggcccaggcaccaccaggagcagcagccagcaccacctacacaggagtcttcagacccagagaaggacggtgccaagggccccaggggcagcaaggccttggtggagcagccagagctgtgcctgctcagcagccagtctcagagaccagcactcagcctcattcagcatgggtccttgatgccagagggccagcaggctcctggctgtgcccagcaggcccagcatgcaggtggtgggacactgggcagcaaggctgctgccggaatcacttctccaatcagtgtttggtgtattatcattttgtgaatttgggtaggggggagggtagggataatttatttttaaataaggttggagatgtcaagttgggttcacttgccatgcaggaagaggcccactagagggcccatcaggcagtgttacctgttagctccctgtggggcaggagtgccaggaccagcctgtaccttgctgtggggctacaggatggtgggcaggatctcaagccagccccctccagctcatgacactgtttggcctttcttggggagaaggcggggtattcccactcaccagccctagctgtcccatggggaaaccctggagccatcccttcggagccaacaagaccgccccagggctatagcagaaagaactttaaagctcaggagggtgacgcccagctccgcctgctgggaagagctcccctccacagctgcagctgatccataggactaccgcaggcccggactcaccaacttgccacatgttctaggtttcagcaacaagactgccaggtggttgggttctgcctttagcctggaccaaagggaagtgaggcccaaggagcttacccaagctgtggcagccgtcccaggccacccccatggaagcaataaagctcttccctgaaaaaaaaaaaaaaaaaaaaaaaaaaaaa

SEQ ID NO: 20 - protein, 189 aa, Homo sapiens
  human ST3 beta-galactoside alpha-2,3-sialyltransferase 3 (ST3Gal-III), isoform k, isoform B1+32
MGLLVFVRNLLLALCLFLVLGFLYYSAWKLHLLQWEEDSNSVVLSFDSAGQTLGSEYDRLGFLLNLDSKLPAELATKYANFSEGACKPGYASALMTAIFPRFSKPAPMFLDDSFRKWARIREFVPPFGIKGQDNLIKAILSVTKEYRLTPALDSLRCRRCIIVGNGGVLANKSLGSRIDDYDIVVRPRL

SEQ ID NO: 21 - DNA, 2550 bp, Mus musculus
  mouse ST3 beta-galactoside alpha-2,3-sialyltransferase 3 (ST3Gal-III) cDNA
ggccgctgcgtccccaccatggcggcgcccgtgcagcttgccgcgtcctgggctccctctggggtcccctcggctgtccccgctattcgcgctgcaattgcctccgcgctctgcgtgcgccctggcgccacccgcccccggcctccctctctgctgcggtaaggcctgccctgcgtccccgaagcagctctcggccgccgcgcccctcccccggccagaccggcccgagccgcgctggggaccaaagcccgggcccgggtgggggccaggcctgccagagggccgcgcgagccgggccgagcgcgccgaggtcacctgtgtgcagtgtcccaggcgcgcgagtctgagcgcaaaggagggtcacggtcacccgggacccgctgtgggacaagcgcaagaaagtccggggaaagcctagcgggagtcaccaagagtagcggttgtcatctctgccttccccggggccagatccttctcggagcgaccgggtcagtttatcaaagtcatgtaggaaattgtgagtcatgtgaagatgggactcttggtatttgtgcgcaatctgctgctagccctctgcctcttcctggtcctgggatttttgtattattctgcctggaagctacacttactccaatgggaagactccaattcattgcttctttcccttgactccgctggacaaaccctaggcacagagtatgataggctgggcttcctcctgaagctggactctaaactgcctgccgagctagccaccaagtacgcaaacttttccgagggagcttgcaaacccggctacgcttcagccatgatgactgccatcttccccaggttctccaagccagcacccatgttcctggatgactcctttcgcaagtgggctaggatccgggagtttgtgccaccttttgggatcaaaggtcaagacaatctgatcaaagccatcttgtcagtcaccaaagaataccgcctgacccctgccttggacagcctccactgccgccgctgcatcattgtaggcaatggaggggtcctcgccaacaagtctctggggtcacggattgacgactatgacattgtgatcagactgaactcagcacctgtgaagggctttgagagggacgtgggcagcaagaccaccctacgcatcacctaccctgagggcgccatgcagcgacctgagcaatatgaacgagactctatctttgtcctcgctggcttcaagtggcaggacttcaagtggctgaagtacatcgtctacaaggagagagtgagtgcatctgatggcttctggaagtccgtggccacccgagtgcccaaggagccccctgagatccgcatcctcaacccatacttcatccaggaggctgccttcactctcattggactgcccttcaacaacggcctcatgggcagagggaacatcccaacccttggcagtgtggcagtgaccatggcactacacggctgtgatgaagtggcagtcgcgggctttggctatgacatgaacacacccaatgcacccctgcactactatgaaactgtgcgcatggcagccatcaaagagtcctggacacacaacatccagcgagagaaagagtttctgcggaagctagtgaaggcacgtgtcatcactgacttaagcagcggtatctgagtgggccggacaccaggccagagaggctcacgccaggaccaagcagctgccacctgcacaggagccttcccacccagagagggacagtgccaagagacccgagggccagtgaggccttggcaaggcaaccagagctgtgcccactgcaggccagcttcagagaccagcaccctgccttgctgcctgggtccatgaagccagagggccaggaggccactggctgtggccagcaggcccagcatgcaggaggtgggacatttggtagcaagaatgctaccagaatcatttctctaatcagtgtttggtgtattatcattttgtgaatttgggtatggggaggggagggataatttatttttaaattagatgtcacatttggtccacttgttgtgcagaaagaggcccactagagggcccatcagacagtggtaccagtgaactccctgaaggatggaggtgtggtgaccatgcttcagggaccgggaggtggatagcatctcatgtcagccatctccagctcacgacactctttggccttccttggggagaagatgaagtactccctctcaccagccaccagtgtcccacaggggaaccccagagccatccctttaaagccaacaggacagctcttggacaagagcagagagaactcaggaggcaaggcccagcccagcctgacgggaatgaatggctccgtccatagctgtccctaatccatggggcagtaatccttcctgaactgtcagatggtctgggtttcttgctgggactgccagctccatgggtcctacccaggaccagagggaagtgaggctccaggacctcacccaggctgccgcagccagccggggcagctctcacggaagcaataaagctctcctctgaatctg

SEQ ID NO: 22 - protein, 374 aa, Mus musculus
  mouse ST3 beta-galactoside alpha-2,3-sialyltransferase 3 (ST3Gal-III)
MGLLVFVRNLLLALCLFLVLGFLYYSAWKLHLLQWEDSNSLLLSLDSAGQTLGTEYDRLGFLLKLDSKLPAELATKYANFSEGACKPGYASAMMTAIFPRFSKPAPMFLDDSFRKWARIREFVPPFGIKGQDNLIKAILSVTKEYRLTPALDSLHCRRCIIVGNGGVLANKSLGSRIDDYDIVIRLNSAPVKGFERDVGSKTTLRITYPEGAMQRPEQYERDSIFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMNTPNAPLHYYETVRMAAIKESWTHNIQREKEFLRKLVKARVITDLSSGI

SEQ ID NO: 23 - DNA, 2257 bp, Mus musculus
  mouse ST3 beta-galactoside alpha-2,3-sialyltransferase 3 (ST3Gal-III) cDNA
gcggaccgcaggattggtggtcccgccgctgcgtccccaccatggcggcgcccgtgcagcttgccgcgtcctgggctccctctggggtcccctcggctgtccccgctattcgcgctgcaattgcctccgcgctctgcgtgcgccctggcgccacccgcccccggcctccctctctgctgcggtcatgtaggaaattgtgagtcatgtgaagatgggactcttggtatttgtgcgcaatctgctgctagccctctgcctcttcctggtcctgggatttttgtattattctgcctggaagctacacttactccaatgggaagactccaattcattgcttctttcccttgactccgctggacaaaccctaggcacagagtatgataggctgggcttcctcctgaagctggactctaaactgcctgccgagctagccaccaagtacgcaaacttttccgagggagcttgcaaacccggctacgcttcagctatgatgactgccatcttccccaggttctccaagccagcacccatgttcctggatgactcctttcgcaagtgggctaggatccgggagtttgtgccaccttttgggatcaaaggtcaagacaatctgatcaaagccatcttgtcagtcaccaaagaataccgcctgacccctgccttggacagcctccactgccgccgctgcatcattgtaggcaatggaggggtcctcgccaacaagtctctggggtcacggattgacgactatgacattgtgatcagactgaactcagcacctgtgaagggctttgagagggacgtgggcagcaagaccaccctacgcatcacctaccctgagggcgccatgcagcgacctgagcaatatgaacgagactctctctttgtcctcgctggcttcaagtggcaggacttcaagtggctgaagtacatcgtctacaaggagagagtgagtgcatctgatggcttctggaagtccgtggccacccgagtgcccaaggagccccctgagatccgcatcctcaacccatacttcatccaggaggctgccttcactctcattggactgcccttcaacaacggcctcatgggcagagggaacatcccaacccttggcagtgtggcagtgaccatggcactacacggctgtgatgaagtggcagtcgcgggctttggctatgacatgaacacacccaatgcacccctgcactactatgaaactgtgcgcatggcagccatcaaagagtcctggacacacaacatccagcgagagaaagagtttctgcggaagctagtgaaggcacgtgtcatcactgacttaagcagcggtatctgagtgggccggacaccgggccagagaggctcacgccaggaccaagcagctgccacctgcacaggagccttcccacccagagagggacagtgccaagagacccgagggccagtgaggccttggcaaggcaaccagagctgtgcccactgcaggccagcttcagagaccagcaccctgccttgctgcctgggtccatgaagccagagggccaggaggccactggctgtggccagcaggcccagcatgcaggaggtgggacatttggtagcaagaatgctaccagaatcatttctctaatcagtgtttggtgtattatcattttgtgaatttgggtatggggaggggagggataatttatttttaaattagatgtcacatttggtccacttgttgtgcagaaagaggcccactagagggcccatcagacagtggtaccagtgaactccctgaaggatggaggtgtggtgaccatgcttcagggaccgggaggtggatagcatctcatgtcagccatctccagctcacgacactctttggccttccttggggagaagatgaagtactccctctcaccagccaccagtgtcccacaggggaaccccagagccatccctttaaagccaacaggacagctcttggacaagagcagagagaactcaggaggcaaggcccagcccagcctgacgggaatgaatggctccgtccatagctgtccctaatccatggggcagtgatccttcctgaactgtcagatggtctgggtttcttgctgggactgccagctccatgggtcctacccaggaccagagggaggtgaggctccaggacctcacccaggctgccgcagccagccggggcagctctcacggaagcaataaagctctcctctgaaaaaaaaaaaaaaaaaaaaaaaaaaaaaaaaaa

SEQ ID NO: 24 - protein, 374 aa, Mus musculus
  mouse ST3 beta-galactoside alpha-2,3-sialyltransferase 3 (ST3Gal-III)
MGLLVFVRNLLLALCLFLVLGFLYYSAWKLHLLQWEDSNSLLLSLDSAGQTLGTEYDRLGFLLKLDSKLPAELATKYANFSEGACKPGYASAMMTAIFPRFSKPAPMFLDDSFRKWARIREFVPPFGIKGQDNLIKAILSVTKEYRLTPALDSLHCRRCIIVGNGGVLANKSLGSRIDDYDIVIRLNSAPVKGFERDVGSKTTLRITYPEGAMQRPEQYERDSLFVLAGFKWQDFKWLKYIVYKERVSASDGFWKSVATRVPKEPPEIRILNPYFIQEAAFTLIGLPFNNGLMGRGNIPTLGSVAVTMALHGCDEVAVAGFGYDMNTPNAPLHYYETVRMAAIKESWTHNIQREKEFLRKLVKARVITDLSSGI

SEQ ID NO: 25 - DNA, 23 bp, Artificial Sequence
  Description of Artificial SequencePCR oligonucleotide primer LE-110 to detect ST3Gal-III wild type and delta alleles
ccagccagcagaggatctgatac

SEQ ID NO: 26 - DNA, 20 bp, Artificial Sequence
  Description of Artificial SequencePCR oligonucleotide primer LE-115 to detect ST3Gal-III wild type allele
cgcagggggcgtttctagac

SEQ ID NO: 27 - DNA, 21 bp, Artificial Sequence
  Description of Artificial SequencePCR oligonucleotide primer rlox to detect ST3Gal-III delta allele
ctcgaattgatccccgggtac

(19) United States
(12) Patent Application Publication
(10) Pub. No.: US 2009/0169535 A1
(43) Pub. Date: Jul. 2, 2009

(54) TREATMENT OF EPILEPSY BY EXPRESSING ST3GAL-III

(75) Inventor: Jamey D. Marth, San Diego, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Regents of the University of California, Oakland, CA (US)

(21) Appl. No.: 11/658,830

(22) PCT Filed: Jul. 27, 2005
(86) PCT No.: PCT/US2005/026671

§ 371 Date: Sep. 24, 2008

Related U.S. Application Data
(60) Provisional application No. 60/591,720, filed on Jul. 27, 2004.

(51) Int. Cl. A61K 38/43; C12Q 1/48; A61K 31/7088; C12N 15/87; A61P 25/00; A61K 31/7105; C12Q 1/68

(52) U.S. Cl. 424/94.5; 435/15; 435/6; 514/44; 435/455

(57) ABSTRACT

The present invention provides methods of diagnosing epilepsy in a mammal by detecting ST3 β-galactoside α-2,3-sialyltransferase 3 (ST3Gal-III) activity or by detecting for one or more mutations in a ST3Gal-III gene that decrease that activity of a ST3Gal-III polypeptide. The invention further provides methods of treating an epileptic condition associated with decreased ST3Gal-III activity by administering one or more agents that increase the activity of ST3Gal-III. Also provided are methods of identifying one or more agents that increase the activity of ST3Gal-III.

### CROSS-REFERENCES TO RELATED APPLICATIONS

This application claims the benefit of U.S. Provisional Application No. 60/591,720, filed Jul. 27, 2004, the entire disclosure of which is hereby incorporated herein by reference in its entirety for all purposes.

### STATEMENT AS TO RIGHTS TO INVENTIONS MADE UNDER FEDERALLY SPONSORED RESEARCH AND DEVELOPMENT

This invention was made with Government support under Grant Nos. DK48247 and HL57345, awarded by the National Institutes of Health. The Government has certain rights in this invention.

### FIELD OF THE INVENTION

The present invention relates to the prevention or treatment of an epileptic condition associated with a ST3Gal-III deficiency. The invention further relates to the identification of agents used to prevent or treat an epileptic condition associated with a ST3Gal-III deficiency by increasing ST3Gal-III activity.

### BACKGROUND OF THE INVENTION

Epilepsy refers to a disorder of brain function characterized by the periodic and unpredictable occurrence of seizures (see, Chapter 21 of Hardman and Limbird, Goodman & Gilman's The Pharmacological Basis of Therapeutics, 10th Edition, 2001, McGraw-Hill; and Chapter 348 of Kaspar, et al, Harrison's Principles of Internal Medicine, 16th Edition, 2005, McGraw-Hill. The various forms of epilepsy or “epilepsy syndromes” have disparate causes. Several genes have been associated with the presence of an epilepsy syndrome, including CHRNA4, CHRNB2, KCNQ2, KCNQ3, SCN1B, LGI1, CSTB, EPM2A, and Doublecortin.

ST3 β-galactoside α-2,3-sialyltransferase 3 (ST3Gal-III) is a sialyltransferase that catalyzes the transfer of a sialic acid to either a sialylate type 1 (Galβ1-3GlcNAc) or a type 2 (Galβ1-4GlcNAc) oligosaccharide to produce a Siaα2-3Galβ1-3GlcNAc or a Siaα2-3Galβ1-4GlcNAc oligosaccharide moiety, respectively. The Siaα2-3Galβ1-3/4GlcNAc moieties produced by ST3Gal-III can be further substituted, for example, with fucosyl moieties, to produce selectin (i.e. E-, P- or L-selectin) ligand structures. ST3Gal-III is widely expressed in different tissues, including in embryonic stem cells, brain, heart, kidney, liver, colon, skeletal muscle, and ovary (see, Ellies, et al., Blood (2002) 100:3618-3625). This invention is based, in part, on the discovery that a deficiency of ST3Gal-III activity in a mammal results in symptoms of epilepsy.

### BRIEF SUMMARY OF THE INVENTION

The invention provides methods of treating epilepsy by upregulating expression of ST3Gal-III. For example, the invention provides 1) methods to treat epilepsy by delivering the gene or gene product for ST3Gal-III, 2) methods for diagnosing epilepsy by identifying and detecting mutations in the ST3Gal-III gene, 3) animal models for epilepsy, and 4) methods for screening compounds for the treatment of epilepsy using the animal models.

Accordingly, in a first aspect, the present invention provides methods for detecting an epileptic condition associated with decreased ST3Gal-III activity in a mammal, the methods comprising detecting ST3Gal-III activity in a sample from the mammal.

The invention further provides methods for detecting an epileptic condition in a mammal by detecting a carbohydrate structure in a sample from the mammal. In this case, the epileptic condition may or may not result from decreased ST3Gal-III activity.

In a related aspect, the invention provides methods for identifying an increased risk for an epileptic condition associated with decreased ST3Gal-III activity, the method comprising identifying one or more mutations in a ST3Gal-III gene, wherein the one or more mutations decrease the activity of the encoded ST3Gal-III polypeptide.

In a further aspect, the invention provides for methods of treating an epileptic condition associated with decreased ST3Gal-III activity in a mammal, the method comprising administering to the mammal a therapeutically effective amount of one or more agents that increase the activity of ST3Gal-III in the mammal.

The invention further provides methods for increasing the levels of one or more Siaα2-3Galβ1-3/4GlcNAc moieties in a central nervous system (CNS) cell, the method comprising, introducing into a CNS cell an expression vector comprising a nucleic acid that encodes a ST3Gal-III polypeptide or enzymatically active fragment thereof.

The invention also provides methods of identifying one or more agents for the treatment of an epileptic condition associated with decreased ST3Gal-III activity in an individual, the method comprising identifying a candidate agent that increases the activity of ST3Gal-III.

### BRIEF DESCRIPTION OF THE DRAWINGS

FIG. 1 illustrates a sialyltransferase reaction of ST3Gal-III sialyltransferase.

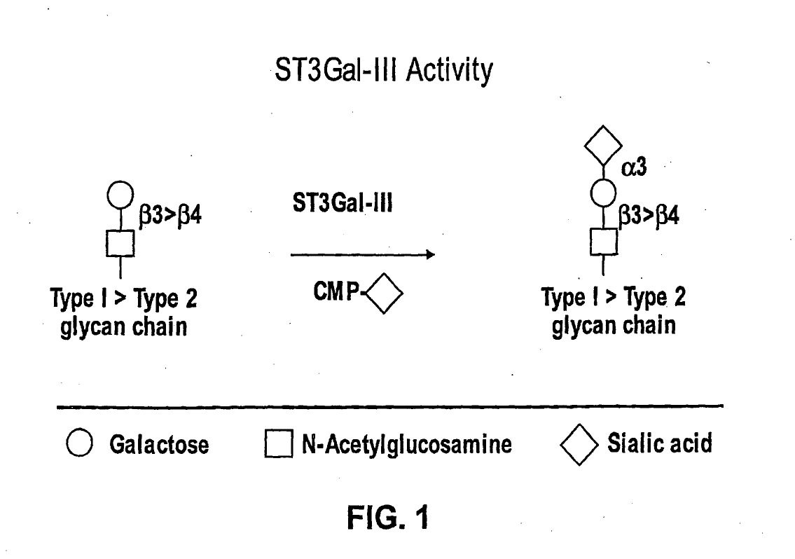

FIG. 2 illustrates the ST3Gal-III sialyltransferase deficiency alters locomotor activity and the contextual fear response.

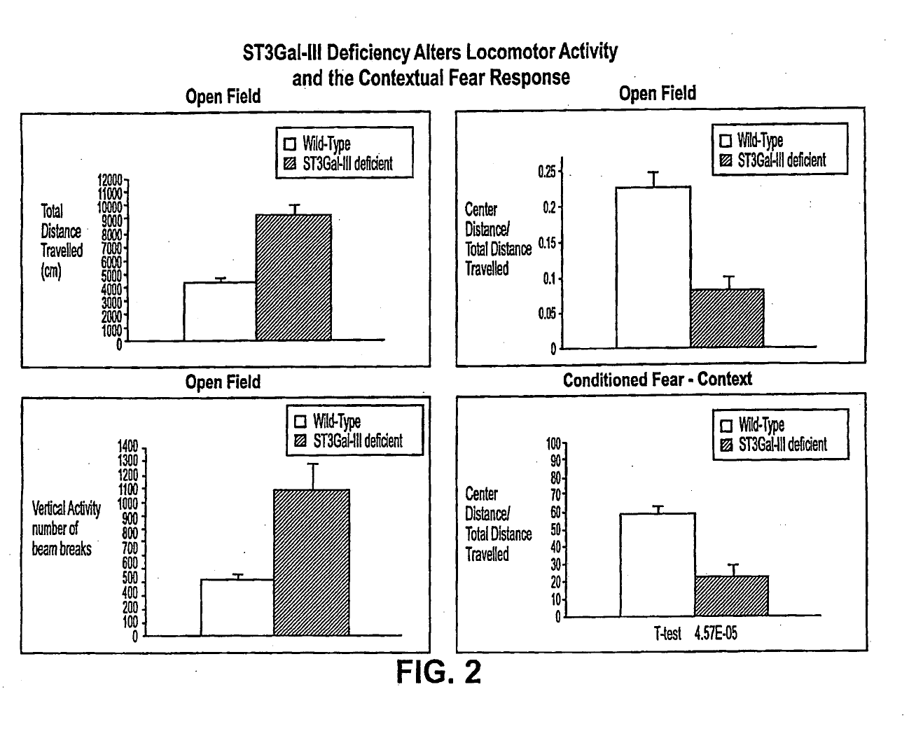

FIG. 3 illustrates impairments in muscular strength and coordination among ST3Gal-III sialyltransferase deficient mice.

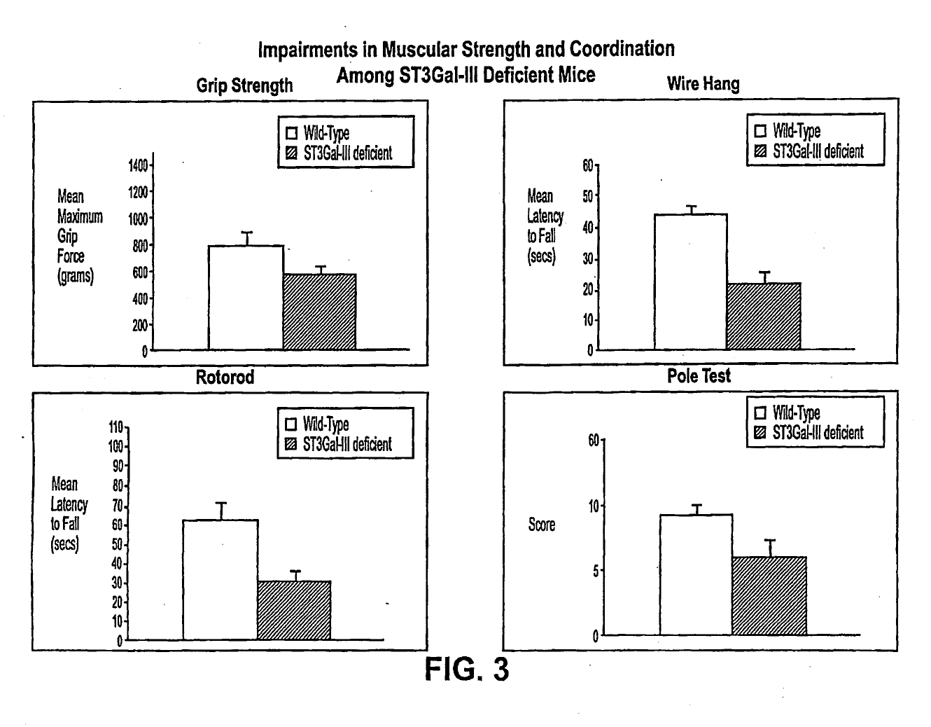

FIG. 4 illustrates increased metabolism in ST3Gal-III sialyltransferase deficient mice.

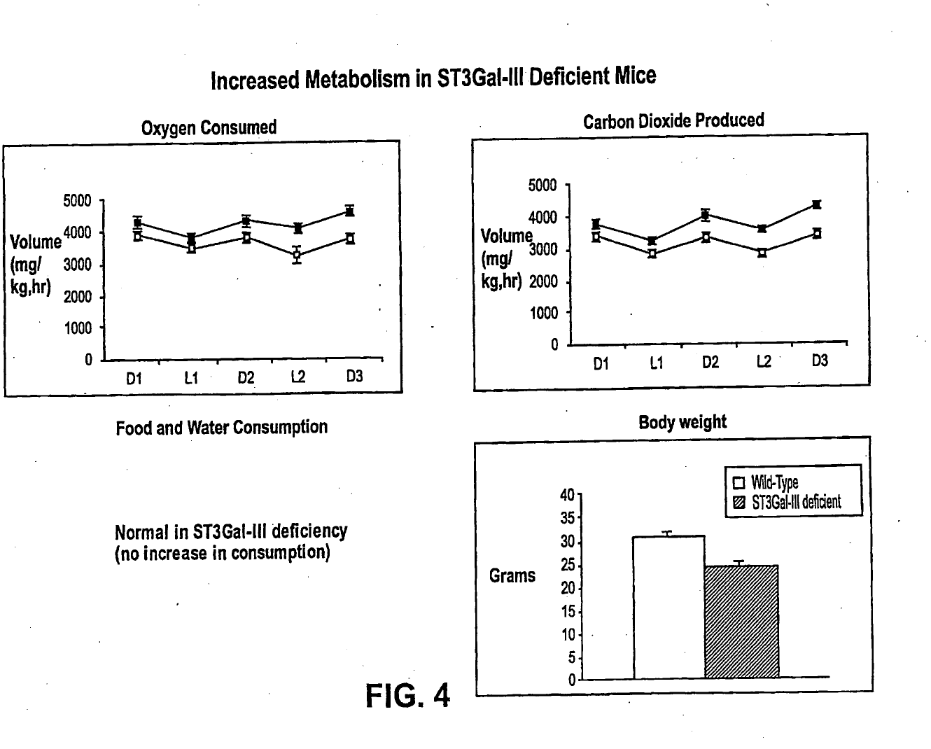

FIG. 5 illustrates that ST3Gal-III sialyltransferase deficiency induces seizures.

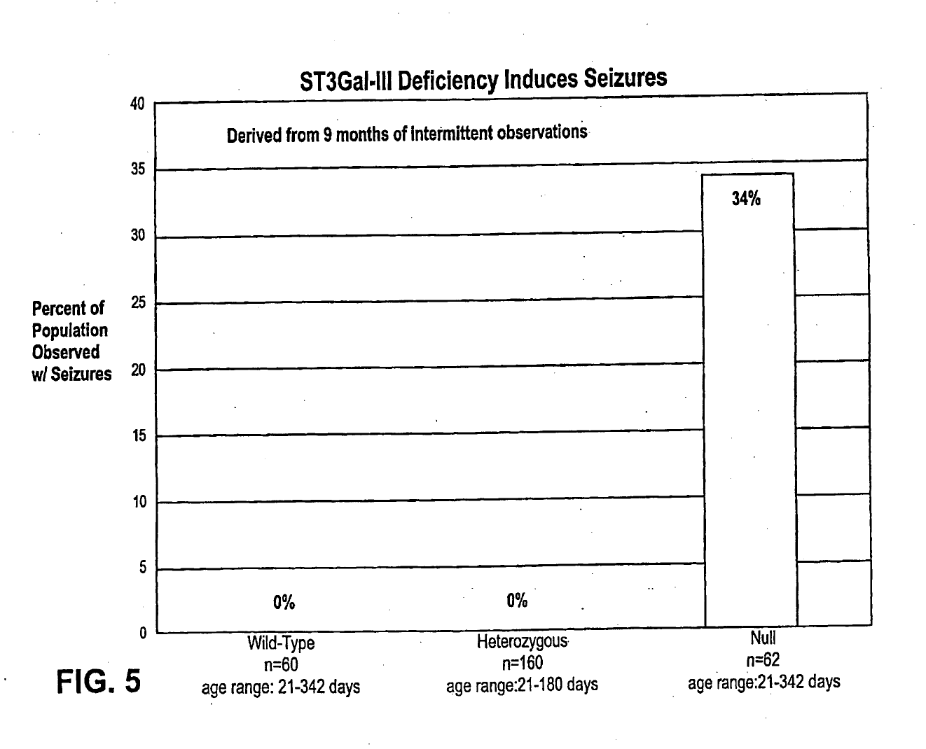

FIG. 6 illustrates that ST3Gal-III sialyltransferase deficiency induces early adult age of seizure onset.

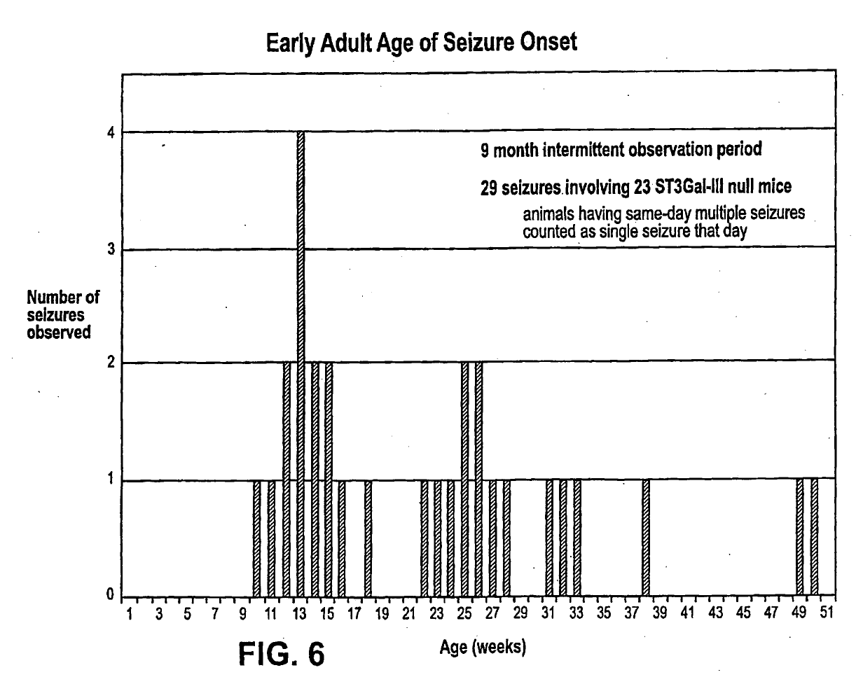

FIG. 7 illustrates the seizure state in ST3Gal-III sialyltransferase deficient mice. ST3Gal-III sialyltransferase deficient mice in a seizure state display a raised tail and splayed limbs.

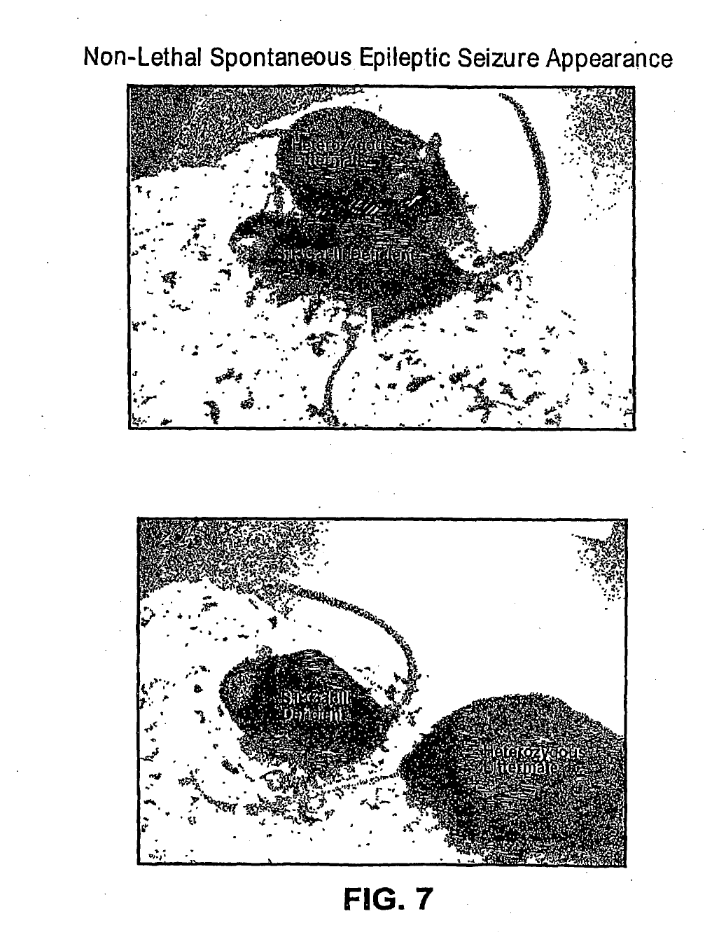

### DETAILED DESCRIPTION

### Definitions

“Epilepsy” or “epileptic condition” refers to a disorder of brain function characterized by the periodic and unpredictable occurrence of seizures (see, Goodman & Gilman's The Pharmacological Basis of Therapeutics, and Harrison's Principles of Internal Medicine, supra).

Epilepsy or an epileptic condition “associated with” a ST3Gal-III deficiency or decrease ST3Gal-III activity includes those epileptic conditions directly or indirectly resulting from decreased ST3Gal-III presence or activity, for example, due to a mutation in a ST3Gal-III gene, abnormally low transcription of ST3Gal-III mRNA, abnormally low translation of a ST3Gal-III polypeptide, or a post-translational mutation in a ST3Gal-III polypeptide.

As used herein, “ST3Gal-III” refers to all mammalian, including human, isoforms and variants of an α2-3 sialyltransferase that catalyzes the transfer of a sialic acid moiety to either a sialylate type 1 (Galβ1-3 GlcNAc) or a type 2 (Galβ1-4GlcNAc) oligosaccharide to produce a Siaα2-3Galβ1-3GlcNAc or a Siaα2-3Galβ1-4GlcNAc oligosaccharide moiety, respectively. These are known in the art. Accordingly, “ST3Gal-III polypeptides” refer to naturally-occurring sequences such as the exemplary sequences provided in SEQ ID NOs:2, 4, 6, 8, 10, 12, 14, 16, 18, 20, 22 or 24, as well as variants or specific fragments thereof, that have α2-3 sialyltransferase activity. Exemplary human and mouse nucleic acid and protein sequences are provided in SEQ ID NOs:1-24. A “ST3Gal-III” polypeptide for use in the invention therefore refers to a polypeptide that: (1) has an amino acid sequence that has greater than about 60% amino acid sequence identity, 65%, 70%, 75%, 80%, 85%, 90%, preferably 91%, 92%, 93%, 94%, 95%, 96%, 97%, 98% or 99% or greater amino acid sequence identity, preferably over a window of at least about 25, 50, 100, 200, or 500, or more amino acids, to one or more amino acid sequences of SEQ ID NOs:2, 4, 6, 8, 10, 12, 14, 16, 18, 20, 22 or 24; (2) bind to antibodies raised against an immunogen comprising an amino acid sequence of SEQ ID NOs:2, 4, 6, 8, 10, 12, 14, 16, 18, 20, 22 or 24; (3) or have at least 15 contiguous amino acids, more often, at least 20, 25, 30, 35, 40, 50 or 100, 200, 300, 400, or 500 contiguous amino acids, of SEQ ID NOs:2, 4, 6, 8, 10, 12, 14, 16, 18, 20, 22 or 24. Similarly, a “ST3Gal-III” polynucleotide for use in the invention therefore refers to a polynucleotide that: (1) has a nucleic acid sequence that has greater than about 60% nucleic acid sequence identity, 65%, 70%, 75%, 80%, 85%, 90%, preferably 91%, 92%, 93%, 94%, 95%, 96%, 97%, 98% or 99% or greater nucleic acid sequence identity, preferably over a window of at least about 25, 50, 100, 200, 300, 400, 500, or more contiguous nucleic acids, to one or more nucleic acid sequences of SEQ ID NOs:1, 3, 5, 7, 9, 11, 13, 15, 17, 19, 21 or 23.

A number of transcripts from the human ST3Gal-III gene have been identified and cloned. Grahn et al. Glycoconj J. 2002 March; 19 (3):197-210 (see Genbank accession numbers NM_{—}174963-174972 for human coding sequences and NM_{—}009176 and BC006710 for mouse coding sequences).

The following abbreviations are used herein:

Gal=galactosyl;

### GlcNAc=N-acetylglucosyl;

Fuc=fucosyl
Sia=sialyl, sialic acid.

Oligosaccharides are considered to have a reducing end and a non-reducing end, whether or not the saccharide at the reducing end is in fact a reducing sugar. In accordance with accepted nomenclature, oligosaccharides are depicted herein with the non-reducing end on the left and the reducing end on the right.

All oligosaccharides described herein are described with the name or abbreviation for the non-reducing saccharide (e.g., Gal), followed by the configuration of the glycosidic bond (α or β), the ring position of the reducing saccharide involved in the bond, and then the name or abbreviation of the reducing saccharide (e.g., GlcNAc). The linkage between two sugars may be expressed, for example, as Galβ4GlcNac. Each saccharide is a pyranose. Glycoside linkages described herein are assumed to originate from the C1 hydroxyl group except for sialic acids, which are linked form the C2 hydroxyl.

An “activator” or “agonist” in the context of this invention generally refers to an agent that binds to, increases, facilitates, enhances activation, e.g., by enhancing sialyl transferase activity. In some embodiments, an “activator” or “agonist” increases the activity or expression of a glycosyltransferase, e.g, a ST3Gal-III.

As used herein, the “central nervous system” or “CNS” refers to tissues and cells of the brain and spinal cord.

“Antibody,” as used herein, refers to a polypeptide substantially encoded by an immunoglobulin gene or immunoglobulin genes, or fragments thereof that specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv, humanized antibodies, chimeric antibodies, etc.).

The term “nucleic acid” or “polynucleotide” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The term “recombinant” when used with reference to a cell indicates that the cell replicates a heterologous nucleic acid, or expresses a peptide or protein encoded by a heterologous nucleic acid. Recombinant cells can contain genes that are not found within the native (non-recombinant) form of the cell. Recombinant cells can also contain genes found in the native form of the cell wherein the genes are modified and re-introduced into the cell by artificial means. The term also encompasses cells that contain a nucleic acid endogenous to the cell that has been modified without removing the nucleic acid from the cell; such modifications include those obtained by gene replacement, site-specific mutation, and related techniques.

A “heterologous sequence” or a “heterologous nucleic acid”, as used herein, is one that originates from a source foreign to the particular host cell, or, if from the same source, is modified from its original form.

A “subsequence” refers to a sequence of nucleic acids or amino acids that comprise a part of a longer sequence of nucleic acids or amino acids (e.g., polypeptide) respectively.

A “recombinant expression cassette” or simply an “expression cassette” is a nucleic acid construct, generated recombinantly or synthetically, with nucleic acid elements that are capable of affecting expression of a structural gene in hosts compatible with such sequences. Expression cassettes include at least promoters and optionally, transcription termination signals as well as other desired sequences that influence gene expression.

The term “isolated” is meant to refer to material which is substantially or essentially free from components which normally accompany the enzyme as found in its native state. Thus, the enzymes of the invention do not include materials normally resulting from their in situ environment. Typically, isolated proteins of the invention are at least about 80% pure, usually at least about 90%, and preferably at least about 95% pure as measured by band intensity on a silver stained gel or other method for determining purity. Protein purity or homogeneity can be indicated by a number of means known in the art, such as polyacrylamide gel electrophoresis of a protein sample, followed by visualization upon staining. For certain purposes high resolution will be needed and HPLC or a similar means for purification utilized.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers. As used herein, the terms encompass amino acid chains of any length, including full-length proteins (i.e., antigens), wherein the amino acid residues are linked by covalent peptide bonds.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified in naturally-occurring cells, e.g., hydroxyproline, γ-carboxyglutamate, and O-phosphoserine. Amino acid “analogs” refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but which functions in a manner similar to a naturally occurring amino acid.

Amino acids may be referred to herein by either the commonly known three letter symbols or by the one-letter symbols recommended by the IUPAC-IUB Biochemical Nomenclature Commission. Nucleotides, likewise, may be referred to by their commonly accepted single-letter codes.

With reference to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are known in the art. Such conservatively modified variants are in addition to and do not exclude polymorphic variants, interspecies homologs, and alleles of the invention.

The following eight groups each contain amino acids that are conservative substitutions for one another:

### 1) Alanine (A), Glycine (G);

2) Aspartic acid (D), Glutamic acid (E);

### 3) Asparagine (N), Glutamine (Q);

### 4) Arginine (R), Lysine (K);

### 5) Isoleucine (I), Leucine (L), Methionine (M), Valine (V);

### 6) Phenylalanine (F), Tyrosine (Y), Tryptophan (W);

### 7) Serine (S), Threonine (T); and

### 8) Cysteine (C), Methionine (M)

(see, e.g., Creighton, Proteins (1984)).

“Percentage of sequence identity” is determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The terms “identical” or percent “identity,” in the context of two or more nucleic acids or polypeptide sequences, refer to two or more sequences or subsequences that are the same.

The term “substantially identical” refers to two or more sequences that have a specified percentage of amino acid residues or nucleotides that are the same (i.e., 60% identity, optionally 65%, 70%, 75%, 80%, 85%, 90%, or 95% identity over a specified region or over an entire sequence when no region is specified), when compared and aligned for maximum correspondence over a comparison window, or designated region as measured using one of the following sequence comparison algorithms or by manual alignment and visual inspection. Optionally, the identity exists over a region that is at least about 25 nucleotides or amino acids in length, or more preferably over a region that is 50, 100, 200, 500 or more nucleotides or amino acids in length.

The present invention provides polynucleotides and polypeptides substantially identical to SEQ ID NOs:1, 3, 5, 7, 9, 11, 13, 15, 17, 19, 21, 23; and 2, 4, 6, 8, 10, 12, 14, 16, 18, 20, 22 or 24, respectively. Thus, a ST3Gal-III polypeptide that is substantially identical, e.g., to one or more of SEQ ID NOs: 2, 4, 6, 8, 10, 12, 14, 16, 18, 20, 22 or 24, typically has an amino acid sequence that is at least 60% identical, often at least 65%, 70%, 75%, 80%, 85%, or 90% identical; and preferably at least 91%, 92%, 93%, 94%, 95%, 96%, 97%, 98% or 99% identical; to one or more of SEQ ID NOs: 2, 4, 6, 8, 10, 12, 14, 16, 18, 20, 22 or 24. Similarly, a ST3Gal-III polynucleotide that is substantially identical, e.g., to one or more of SEQ ID NOs: 1, 3, 5, 7, 9, 11, 13, 15, 17, 19, 21 or 23, typically has a nucleic acid sequence that is at least 60% identical, often at least 65%, 70%, 75%, 80%, 85%, or 90% identical; and preferably at least 91%, 92%, 93%, 94%, 95%, 96%, 97%, 98% or 99% identical; to one or more of SEQ ID NOs: 1, 3, 5, 7, 9, 11, 13, 15, 17, 19, 21 or 23. The percent identity is preferably over a window of at least 50, 100, 200, 300, 500 or more nucleic acids or amino acids, or over the complete length of the sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are known in the art. Optimal alignment of sequences for comparison can be conducted, e.g., by the local homology algorithm of Smith and Waterman (1970) Adv. Appl. Math. 2:482c, by the homology alignment algorithm of Needleman and Wunsch (1970) J. Mol. Biol. 48:443, by the search for similarity method of Pearson and Lipman (1988) Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., Current Protocols in Molecular Biology (1995 supplement)).

An example of an algorithm that is suitable for determining percent sequence identity and sequence similarity are the BLAST and BLAST 2.0 algorithms, which are described in Altschul et al. (1977) Nuc. Acids Res. 25:3389-3402, and Altschul et al. (1990) J. Mol. Biol. 215:403-410, respectively. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, optionally 10 times background hybridization. Exemplary stringent hybridization conditions can be as following: 50% formamide, 5×SSC, and 1% SDS, incubating at 42° C., or 5×SSC, 1% SDS, incubating at 65° C., with wash in 0.2×SSC, and 0.1% SDS at 55° C., 60° C., or 65° C. Such washes can be performed for 5, 15, 30, 60, 120, or more minutes.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Exemplary “moderately stringent hybridization conditions” include a hybridization in a buffer of 40% formamide, 1 M NaCl, 1% SDS at 37° C., and a wash in 1×SSC at 45° C. Such washes can be performed for 5, 15, 30, 60, 120, or more minutes. A positive hybridization is at least twice background. Those of ordinary skill will readily recognize that alternative hybridization and wash conditions can be utilized to provide conditions of similar stringency.

“Activators” of expression or of activity are used to refer to activating, stimulating, enhancing molecules, respectively, identified using in vitro and in vivo assays for expression or activity. Samples or assays comprising a polypeptide of interest, e.g., a glycosyltransferase, e.g. ST3Gal-III, that are treated with a potential agonist can be compared to control samples without the agonist to examine the extent of effect. Control samples (untreated with agonists) are assigned a relative activity value of 100%. Activation of the polypeptide is achieved when the polypeptide activity value relative to the control is 110%, optionally 150%, optionally 200%, 300%, 400%, 500%, or 1000-3000% or more higher activity.

A “therapeutically effective amount”, “pharmacologically acceptable dose”, “pharmacologically acceptable amount” means that a sufficient amount of a ST3Gal-III activity enhancer is present to achieve a desired result, e.g., activating, agonizing, increasing, enhancing the sialyltransferase activity of ST3Gal-III.

### DETAILED EMBODIMENTS

In a first aspect, the present invention provides methods for detecting an epileptic condition associated with decreased ST3Gal-III activity in a mammal, the methods comprising detecting ST3Gal-III activity in a sample from the mammal. ST3Gal-III polypeptides and their activity can be detected using standard assays (e.g., polypeptides can be detected using antibodies; activity can be detected using standard enzymology techniques). An epileptic condition, whether or not associated with a decrease in ST3Gal-III activity, can be diagnosed by detecting the levels of carbohydrate structures used or made by ST3Gal-III, for example, by detecting decreasing levels of substrate (e.g., an activated sialic acid or a Galβ1-3/4GlcNAc) or increasing levels of product (e.g., a Siaα2-3Galβ1-3GlcNAc or a Siaα2-3Galβ1-4GlcNAc oligosaccharide moiety). Carbohydrate structures can be detected directly (e.g., using antibodies, high performance liquid chromatography (HPLC), NMR) or indirectly (e.g., by detecting binding to one or more antibodies, lectins (i.e., Maackia Amurensis II (MAL II)), selectins, CD33, or sialoadhesin). ST3Gal-III activity additionally can be detected by detecting an increase or decrease in the transcription of a ST3Gal-III coding sequence or translation of a ST3Gal-III polypeptide.

The invention further provides methods of detecting an epileptic condition, the method comprising detecting one or more carbohydrate structures in a sample from the mammal. In cases where the epileptic condition results from decreased ST3Gal-III activity, the carbohydrate structure can be a product of ST3Gal-III, for example, one or more of a Siaα2-3Galβ1-3GlcNAc or a Siaα2-3Galβ1-4GlcNAc oligosaccharide moiety. The carbohydrate structure can also be one or more of a substrate of ST3Gal-III, for example, a Galβ1-3GlcNAc or a Galβ1-4GlcNAc oligosaccharide moiety. The carbohydrate structure can be detected using methods known in the art, as described herein. In some embodiments the presence of the carbohydrate structure is abnormally high or abnormally low in comparison to a the presence of the carbohydrate structure in a sample from a mammal not suffering an epileptic condition. In some embodiments, the one or more carbohydrate structures are detected on a CNS cell, including a brain cell.

In a related aspect, the invention provides methods for identifying an increased risk for an epileptic condition associated with decreased ST3Gal-III activity, the method comprising identifying one or more mutations in a ST3Gal-III gene, wherein the one or more mutations decrease the activity of the encoded ST3Gal-III polypeptide. The decrease in ST3Gal-III activity of a ST3Gal-III polypeptide expressed from a mutant gene can be made in comparison to the activity of a ST3Gal-III polypeptide expressed from a wild-type gene. The one or more mutations can be in an active site of the ST3Gal-III enzyme, and typically occur in regions of the enzyme highly conserved among mammals. Such highly conserved regions can be identified by aligning nucleic acid and/or amino acid sequences of known ST3Gal-III sequences from different mammalian species, for example, from human, mouse, rat, cow, pig and hamster. This can be done using readily available algorithms known in the art (e.g., BLAST; Lipman and Pearson's Align program).

In a further aspect, the invention provides for methods of treating an epileptic condition associated with decreased ST3Gal-III activity in a mammal, the method comprising administering to the mammal a therapeutically effective amount of one or more agents that increase the activity of ST3Gal-III in the mammal. In some embodiments, the agent is a nucleic acid encoding an enzymatically active ST3Gal-III or an enzymatically active fragment thereof. The agent that increases the activity of ST3Gal-III also can be a ST3Gal-III polypeptide. In some embodiments, the agent increases the level of ST3Gal-III protein expression, or increases the level of ST3Gal-III mRNA expression.

The invention further provides methods for increasing the levels of one or more Siaα2-3Galβ1-3/4GlcNAc moieties in a central nervous system (CNS) cell, the method comprising, introducing into a CNS cell an expression vector comprising a nucleic acid that encodes a ST3Gal-III polypeptide or enzymatically active fragment thereof. In some embodiments, the CNS cell is a differentiated brain cell. In some embodiments, the CNS cell is an undifferentiated stem cell. The expression vector can be introduced into the CNS cell either ex vivo or in vivo.

The invention also provides methods of identifying one or more agents for the treatment of an epileptic condition associated with decreased ST3Gal-III activity in an individual, the method comprising identifying a candidate agent that increases the activity of ST3Gal-III. In some embodiments, the agent that increases the activity of ST3Gal-III is identified by contacting the candidate agent with a ST3Gal-III polypeptide or enzymatically active fragment thereof. In some embodiments, the agent that increases the activity of ST3Gal-III is identified by contacting the candidate agent with a substrate (e.g., an activated sialic acid or a Galβ1-3/4GlcNAc) or a product (e.g., a Siaα2-3Galβ1-3GlcNAc or a Siaα2-3Galβ1-4GlcNAc oligosaccharide moiety) of a ST3Gal-III. Candidate agents of interest increase the amount of Siaα2-3Galβ1-3GlcNAc or Siaα2-3Galβ1-4GlcNAc moieties in a CNS cell, particularly a brain cell.

### Identification of ST3Gal-III Activity Enhancers

A number of different screening protocols can be utilized to identify agents that increase the level of expression or activity of ST3Gal-III in cells, particularly mammalian cells, and especially human cells. In general terms, the screening methods involve screening a plurality of agents to identify an agent that increases the activity of ST3Gal-III.

This invention involves routine techniques in the field of recombinant genetics. Basic texts disclosing the general methods of use in this invention include Sambrook et al., Molecular Cloning, A Laboratory Manual (3rd ed. 2001); Kriegler, Gene Transfer and Expression: A Laboratory Manual (1990); and Current Protocols in Molecular Biology (Ausubel et al., eds., 1994-2005)).

Activators of ST3Gal-III, e.g., ST3Gal-III agonists (i.e., agents that increase the activity or expression of ST3Gal-III) are useful for increasing or replacing ST3Gal-III sialyltransferase activity in individuals having an epileptic condition associated with deficient ST3Gal-III activity.

### ST3Gal-III Activating Agents

ST3Gal-III activating agents can be any small chemical compound, or a biological entity, such as a protein, sugar, nucleic acid or lipid.

A wide variety of methods can be used to identify agents that increase ST3Gal-III activity or level. Typically, test compounds will be small chemical molecules and/or peptides. Essentially any chemical compound can be used as a potential activity enhancer. In the assays of the invention, although most often compounds that can be dissolved in aqueous or organic (especially DMSO-based) solutions are used. The assays can be designed to screen large chemical libraries by automating the assay steps and providing compounds from any convenient source to assays, which are typically run in parallel (e.g., in microtiter formats on microtiter plates in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs, Switzerland) and the like.

In some embodiments, high throughput screening methods involve providing a combinatorial chemical or peptide library containing a large number of potential therapeutic compounds (potential activity enhancing compounds). Such “combinatorial chemical libraries” or “ligand libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptoids (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al. J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14 (3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, January 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, U.S. Pat. No. 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

### ST3Gal-III Polypeptides

ST3Gal-III sequences are highly conserved amongst mammals. For example, mouse and human ST3Gal-III polypeptides share about 90-95% sequence identity (isoform B1 shares about 97% identity). Mouse and human ST3Gal-III encoding polynucleotide sequences share about 70% sequence identity. ST3Gal-III nucleic acid and amino acid sequences for other mammalian species, including pig, cow and hamster, also have been identified.

ST3Gal-III polypeptides for use in the invention include ST3Gal-III polypeptides comprising: a naturally-occurring amino acid sequence including a human ST3Gal-III (e.g., one or more of SEQ ID NOs:2, 4, 6, 8, 10, 12, 14, 16, 18 or 20), or other animal ortholog, e.g., mouse ST3Gal-III (e.g., SEQ NOs:22 or 24). Other naturally occurring variants (relative to the exemplary sequences provided herein); or engineered ST3Gal-III polypeptides can also be employed. Accordingly, substantial information is available to those of skill in identifying and/or generating such variant ST3Gal-III polypeptides.

ST3Gal-III polypeptide variants for use in the invention can be tested, e.g., by assessing the ability of the polypeptide to transfer a sialic acid moiety onto a sialylate type 1 (Galβ1-3GlcNAc) or a type 2 (Galβ1-14GlcNAc) oligosaccharide. Such ST3Gal-III polypeptides can then be used in assays to identify activators of ST3Gal-III activity.

### Methods of Screening for Activity Enhancers of ST3Gal-III.

A number of different screening protocols can be utilized to identify agents that enhance the activity of ST3Gal-III.

Screening can be performed using isolated, purified or partially purified reagents. In some embodiments, purified or partially purified ST3Gal-III (e.g., cell fractions comprising a ST3Gal-III polypeptide) can be used.

Alternatively, cell-based methods of screening can be used. For example, cells that naturally-express ST3Gal-III or that recombinantly express ST3Gal-III can be used. In some embodiments, the cells used are mammalian cells, including but not limited to, human cells. In general terms, the screening methods involve screening a plurality of agents to identify an agent that increases the activity of ST3Gal-III by, e.g., binding to and/or increasing the activity of a ST3Gal-III polypeptide, preventing an activator from binding to ST3Gal-III, increasing association of activator with ST3Gal-III, or activating expression of ST3Gal-III.

ST3Gal-III Binding Assays

Optionally, preliminary screens can be conducted by screening for agents that bind to ST3Gal-III. Binding assays are also useful, e.g., for identifying endogenous, or other proteins, that interact with ST3Gal-III. For example, antibodies or other molecules that bind ST3Gal-III can be identified in binding assays. Such antibodies have use, e.g. as diagnostic agents.

Binding assays usually involve contacting a ST3Gal-III protein with one or more test agents and allowing the ST3Gal-III protein and test agent(s) to form a binding complex. Binding complexes that are formed can be detected using any of a number of established analytical techniques. Protein binding assays include, but are not limited to, methods that measure co-precipitation or co-migration on non-denaturing SDS-polyacrylamide gels, and co-migration on Western blots (see, e.g., Bennet, J. P. and Yamamura, H. I. (1985) “Neurotransmitter, Hormone or Drug Receptor Binding Methods,” in Neurotransmitter Receptor Binding (Yamamura, H. I., et al., eds.), pp. 61-89. Other binding assays involve the use of mass spectrometry or NMR techniques to identify molecules bound to ST3Gal-III or displacement of labeled substrates. The ST3Gal-III protein utilized in such assays can be naturally expressed, cloned or synthesized.

In addition, mammalian or yeast two-hybrid approaches (see, e.g., Bartel, P. L. et. al. Methods Enzymol, 254:241 (1995)) can be used to identify polypeptides or other molecules that interact or bind when expressed together in a cell.

ST3Gal-III Activity

ST3Gal-III activators can be identified by screening for agents that alter an activity of ST3Gal-III. Analysis of ST3Gal-III activity is performed according to general biochemical procedures. Such assays include cell-based assays as well as in vitro assays involving purified or partially purified ST3Gal-III polypeptides or cellular fractions comprising ST3Gal-III.

In some embodiments, ST3Gal-III activators are identified by screening a plurality of agents (generally in parallel) for the ability to enhance ST3Gal-III activity. The level of ST3Gal-III activity in a cell or other sample can be determined and compared to a baseline value (e.g., a control value or the ST3Gal-III activity in a sample not contacted with an agent or the ST3Gal-III activity in a sample contacted to a different agent).

ST3Gal-III catalyzes the transfer of a sialic acid moiety to a sialylate type 1 (Galβ1-3GlcNAc) or a type 2 (Galβ1-4GlcNAc) oligosaccharide. Thus, ST3Gal-III activity can be determined using any number of direct and indirect indicators of activity. For example, the transferase activity can be determined directly (e.g., using the standard enzymology techniques, for example, by measuring the increase of a product (i.e., a Siaα2-3Galβ1-3/4GlcNAc) or the decrease of a substrate (i.e., activated sialic acid or a sialylate type 1 or type 2 oligosaccharide). Carbohydrate structures can be detected using methods known in the art, for example, by using antibodies specific for particular oligosaccharide moieties, chromatographic techniques (e.g., HPLC), mass spectrometry, or nuclear magnetic resonance (NMR). For instance, specific lectins, selectins, sialoadhesin, CD33 or antibodies raised against the ligand can be used. Methods suitable for use in the diagnostic methods of the invention described in WO 00/33076. Alternatively, the level of glycosylation can be determined indirectly, typically by measuring the level of lectin or selectin binding, e.g., P-, E- or L-selectin binding. Any lectin or selectin that binds to a Siaα2-3Galβ1-3/4GlcNAc can be used, for example, Maackia Amurensis II (MAL II) lectin.

In some embodiments, cells transiently transfected with ST3Gal-III are measured for ST3Gal-III activity in suspension or adhered to the plate, within an isotonic buffer. The cells are then contacted to one or more agents and tested for ST3Gal-III activity.

Screening methods to identify enhancers of ST3Gal-III activity typically employ in at least one of the steps, an assay that uses a ST3Gal-III polypeptide.

Expression Assays

Screening methods for a compound that increases the expression of ST3Gal-III are also provided. Screening methods generally involve conducting cell-based assays in which test compounds are contacted with one or more cells expressing ST3Gal-III, and then detecting an increase or decrease in ST3Gal-III expression (either transcript, translation product). Assays can be performed with cells that naturally express ST3Gal-III or in cells recombinantly altered to express a ST3Gal-III.

ST3Gal-III expression can be detected in a number of different ways. For example, the expression level of ST3Gal-III in a cell can be determined by evaluating mRNA expression using known methods, e.g., northern blot analysis, in situ hybridization and the like. Alternatively, ST3Gal-III protein can be detected, e.g., using immunological methods, such as ELISA, immunoblotting, immunoprecipitations, and other well-known techniques.

Other cell-based assays involve reporter assays conducted with cells using standard reporter gene assays. These assays can be performed in either cells that do, or do not, express ST3Gal-III. Some of these assays are conducted with a heterologous nucleic acid construct that comprises a ST3Gal-III promoter that is operably linked to a reporter gene that encodes a detectable product. A number of different reporter genes can be utilized, including, green fluorescent protein, and enzyme reporters such as β-glucuronidase, CAT (chloramphenicol acetyl transferase; Alton and Vapnek (1979) Nature 282:864-869), luciferase, β-galactosidase and alkaline phosphatase (Toh, et al. (1980) Eur. J. Biochem. 182:231-238; and Hall et al. (1983) J. Mol. Appl. Gen. 2:101).

In these assays, cells harboring the reporter construct are contacted with a test compound. Increased expression is monitored by detecting the level of a detectable reporter. A number of different kinds of ST3Gal-III expression enhancers can be identified in this assay. For example, a test compound that activates the promoter by binding to it, or by binding to and activating a transcription factor that binds to the promoter, or by inducing a cascade that produces a molecule that activates the promoter, or that otherwise activates the promoter can be identified. Similarly, a test compound that, e.g., enhances the promoter by binding to it, or by binding to a transcription factors or other regulatory factor that results in activating a ST3Gal-III promoter can also be identified.

The level of expression or activity can be compared to a baseline value. The baseline value can be a value for a control sample or a statistical value that is representative of ST3Gal-III expression levels for a control population (e.g., individuals not having or at risk for epilepsy) or cells (e.g., tissue culture cells not exposed to a ST3Gal-III agonist or antagonist). Expression levels can also be determined for cells that do not express a ST3Gal-III as a negative control. Such cells generally are otherwise substantially genetically the same as the test cells.

Various controls can be conducted to ensure that an observed activity is authentic including running parallel reactions with cells that lack the reporter construct or by not contacting a cell harboring the reporter construct with test compound.

Computer-Based Assays

Other assays for compounds that increase the activity of ST3Gal-III involves computer-assisted drug design, in which a computer system is used to generate a three-dimensional structure of ST3Gal-III based on the structural information encoded by its amino acid sequence. The input amino acid sequence interacts directly and actively with a pre-established algorithm in a computer program to yield secondary, tertiary, and quaternary structural models of the protein. The models of the protein structure are then examined to identify regions (e.g., the active site) of the structure that have the ability to bind ligands or otherwise be enhance ST3Gal-III activity. These regions are then used to identify polypeptides that bind to ST3Gal-III.

Once the tertiary structure of a protein of interest has been generated, potential activity enhancers can be identified by the computer system. Three-dimensional structures for potential activity enhancers are generated by entering chemical formulas of compounds. The three-dimensional structure of the potential activity enhancer is then compared to that of ST3Gal-III to identify potential activity enhancer binding sites to ST3Gal-III. Binding affinity between the protein and activity enhancer is determined using energy terms to determine which ligands have an enhanced probability of binding to the protein.

Validation of Candidate Activators

Agents that are initially identified by any of the foregoing screening methods can be further tested to validate the apparent activity and/or determine other biological effects of the agent. In some cases, the identified agent is tested for the ability to increase ST3Gal-III activity and/or the levels of Siaα2-3Galβ1-3GlcNAc or Siaα2-3Galβ1-4GlcNAc oligosaccharide moieties.

In vitro assays using isolated CNS cells, particularly brain cells (normal or epileptic) can be performed in the presence or absence of the candidate activator. In some embodiments, validation studies are conducted with suitable animal models. The basic format of such methods involves administering a lead compound identified during an initial screen to an animal that serves as a model for humans and then determining if ST3Gal-III activity and/or expression, e.g., selectin binding, sialyltransferase activity and the like, is in fact activated following administration of the ST3Gal-III activator. The animals can also be monitored for the increase or decrease of seizure activity by EEG analysis or subject to behavioral monitoring (locomotor activity, contextual fear responses, muscular strength and coordination). The animal models utilized in validation studies generally are mammals of any kind, but usually mice. Specific examples of suitable animals include mice having a knocked-out ST3Gal-III gene or a ST3Gal-III gene having one or more mutations such that the ST3Gal-III sialyltransferase activity is decreased in comparison to a mouse having a wild-type ST3Gal-III gene. Mice genetically deficient for a ST3Gal-III gene are described in Ellies, et al., Blood (2002) 100:3618-25. Other established animal epilepsy models can find use in the screening and validation of ST3Gal-III activity enhancer, whether using the animals themselves or the methods of monitoring for epileptic symptoms, including those described in Yang and Frankel, Adv Exp Med Biol (2004) 548:1-11; Stables, et al., Epilepsia (2002) 43:1410-20; Kupferberg, Epilepsia (2001) 4:7-12; and Seyfried, et al., (1999) 79:279-90.

### Administration and Pharmaceutical Compositions

Activators of ST3Gal-III (e.g., ST3Gal-III agonists) can be administered directly to the mammalian subject in need thereof for activation of ST3Gal-III activity in vivo. Individuals in need of activators of ST3Gal-III include, for example, individuals with an epileptic condition or another seizure disorder associated with deficient ST3Gal-III activity. Administration can be by any of the routes normally used for introducing a compound into ultimate contact with the tissue to be treated and is known to those of skill in the art. Although more than one route can be used to administer a particular composition, a particular route can often provide a more immediate and more effective reaction than another route.

The pharmaceutical compositions of the invention may comprise a pharmaceutically acceptable carrier. Pharmaceutically acceptable carriers are determined in part by the particular composition being administered, as well as by the particular method used to administer the composition. Accordingly, there is a wide variety of suitable formulations of pharmaceutical compositions of the present invention (see, e.g., Remington: The Science and Practice of Pharmacy, 20th ed. 2003)).

Activators, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for administration include aqueous and non-aqueous solutions, isotonic sterile solutions, which can contain antioxidants, buffers, bacteriostats, and solutes that render the formulation isotonic, and aqueous and non-aqueous sterile suspensions that can include suspending agents, solubilizers, thickening agents, stabilizers, and preservatives. The formulations of compounds can be presented in unit-dose or multi-dose sealed containers, such as ampoules and vials. Solutions and suspensions can be prepared from sterile powders, granules, and tablets of the kind previously described. The activators can also be administered as part of a prepared food or drug.

In the practice of this invention, compositions can be administered, for example, orally, nasally, topically, intravenously, intraperitoneally, or intrathecally. Optimally, an activator or a ST3Gal-III polypeptide is delivered to brain tissue. Strategies for delivery of an activator or a polypeptide across the blood-brain barrier include, for example, double-coated poly (butylcyanoacrylate) nanoparticulate delivery systems (Das and Lin, J Pharm Sci. (2005) 94:1343-53); convection-enhanced delivery (Lonser, et al., Ann Neurol. (2005) 57:542-8); ST3Gal-III conjugation to a transferring receptor (TfR)-specific ligand, including an anti-TfR antibody (Zhang and Pardridge, J Pharmacol Exp Ther. (2005) 313:1075-81; see also, U.S. Pat. Nos. 5,977,307 and 5,672,683); formulations which include the amphiphilic block copolymer Pluronic P85 (P85) (Batrakova, et al. Pharm Res. (2004) 21:1993-2000); formulations which include Zonula occludens toxin (Zot) or its biologically active fragment, DeltaG (Salama, et al., J Pharmacol Exp Ther. (2005) 312:199-205); nanoparticles (U.S. Patent Publication No. 2004/0131692) and co-administration with hyaluronidase (U.S. Patent Publication No. 2003/0215432).

The dose administered to a patient, in the context of the present invention should be sufficient to induce a beneficial response in the subject over time. The optimal dose level for any patient will depend on a variety of factors including the efficacy of the specific activator employed, the age, body weight, physical activity, and diet of the patient, on a possible combination with other drugs, and on the severity of the case of epilepsy. If is recommended that the daily dosage of the activator be determined for each individual patient by those skilled in the art in a similar way as for known compositions used to treat epilepsy. The size of the dose also will be determined by the existence, nature, and extent of any adverse side-effects that accompany the administration of a particular compound or vector in a particular subject.

In determining the effective amount of the activator to be administered a physician may evaluate circulating plasma levels of the activator, activator toxicity, and the production of anti-activator antibodies. In general, the dose equivalent of an activator is from about 1 ng/kg to 10 mg/kg for a typical subject.

ST3Gal-III activators can be administered at a rate determined by the LD-50 of the activator, and the side-effects of the activator at various concentrations, as applied to the mass and overall health of the subject. Administration can be accomplished via single or divided doses.

The compounds of the present invention can also be used effectively in combination with one or more additional active agents currently used to treat epilepsy, depending on the desired target therapy, including carbamazepine, phenytoin, valproate, phenobarbital, primidone, and ethosuximide (see, Chapter 21 of Goodman and Gilman's, supra). Combination therapy includes administration of a single pharmaceutical dosage formulation that contains a ST3Gal-III activator of the invention and one or more additional active agents, as well as administration of a ST3Gal-III activator and each active agent in its own separate pharmaceutical dosage formulation. For example, a ST3Gal-III activator and another active agent used to treat epilepsy can be administered to the human subject together in a single oral dosage composition, such as a tablet or capsule, or each agent can be administered in separate oral dosage formulations. Where separate dosage formulations are used, a ST3Gal-III activator and one or more additional active agents can be administered at essentially the same time (i.e., concurrently), or at separately staggered times (i.e., sequentially). Combination therapy is understood to include all these regimens.

Pharmaceutically acceptable carriers are determined in part by the particular composition being administered, as well as by the particular method used to administer the composition. Accordingly, there is a wide variety of suitable formulations of pharmaceutical compositions of the present invention, as described below (see, e.g., Remington: The Science and Practice of Pharmacy, 20th ed., 2003).

### Administration of Nucleic Acid Activators

In one aspect of the present invention, ST3Gal-III activators can also comprise nucleic acid molecules that express ST3Gal-III. Conventional viral and non-viral based gene transfer methods can be used to introduce nucleic acids encoding ST3Gal-III polypeptides in mammalian cells or target tissues, for example CNS tissue or brain tissue. Such methods can be used to administer nucleic acids encoding polypeptides of the invention to cells in vitro. In some embodiments, the nucleic acids encoding polypeptides of the invention are administered for in vivo or ex vivo gene therapy uses. Non-viral vector delivery systems include DNA plasmids, naked nucleic acid, and nucleic acid complexed with a delivery vehicle such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10):1149-1154 (1988); Vigne, Restorative Neurology and Neuroscience 8:35-36 (1995); Kremer & Perricaudet, British Medical Bulletin 51 (1):31-44 (1995); Haddada et al., in Current Topics in Microbiology and Immunology Doerfler and Böhm (eds) (1995); and Yu et al., Gene Therapy 1:13-26 (1994).

Methods of non-viral delivery of nucleic acids encoding engineered polypeptides of the invention include lipofection, microinjection, biolistics, virosomes, liposomes, immunoliposomes, polycation or lipid:nucleic acid conjugates, naked DNA, artificial virions, and agent-enhanced uptake of DNA. Lipofection is described in e.g., U.S. Pat. No. 5,049,386, U.S. Pat. No. 4,946,787; and U.S. Pat. No. 4,897,355) and lipofection reagents are sold commercially (e.g., Transfectam and Lipofectin). Cationic and neutral lipids that are suitable for efficient receptor-recognition lipofection of polynucleotides include those of Felgner, WO 91/17424, WO 91/16024. Delivery can be to cells (ex vivo administration) or target tissues (in vivo administration).

The preparation of lipid:nucleic acid complexes, including targeted liposomes such as immunolipid complexes, is known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of ST3GAL III nucleic acids is known in the art. RNA or DNA viral based systems for the delivery of nucleic acids encoding polypeptides of the invention take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of polypeptides of the invention could include retroviral, lentivirus, adenoviral, adeno-associated and herpes simplex virus vectors for gene transfer. Viral vectors are currently the most efficient and versatile method of gene transfer in target cells and tissues. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, fetal tissue, umbilical tissue, tissue biopsy) or universal donor hematopoietic stem cells, followed by reimplantation of the cells into a patient, usually after selection for cells which have incorporated the vector. Optimally, a nucleic acid encoding a ST3Gal-III or an enzymatically active fragment thereof is delivered to brain tissue. Strategies for delivery of a nucleic acid across the blood brain barrier include, for example, virally transduced bone marrow cells (Makar, et al., Neurosci Lett. (2004) 356:215-9); poly-L-lysine modified iron oxide nanoparticles (IONP-PLL) (xiang, et al., J Gene Med. (2003) 5:803-17); liposomes (U.S. Pat. No. 6,372,250); transfection of one or more neurons which “straddle” the blood-brain barrier (U.S. Patent Publication No. 2003/0083299) and co-administration with hyaluronidase (U.S. Patent Publication No. 2003/0215432). See also, Schlachetzki, et al., Neurology (2004) 62:1275-81.

Ex vivo cell transfection for diagnostics, research, or for gene therapy (e.g., via re-infusion of the transfected cells into the host organism) is known to those of skill in the art. In some embodiments, cells are isolated from the subject organism, transfected with a nucleic acid (gene or cDNA) encoding a polypeptides of the invention, and re-infused back into the subject organism (e.g., patient). Various cell types suitable for ex vivo transfection are known to those of skill in the art (see, e.g., Freshney et al., Culture of Animal Cells, A Manual of Basic Technique (3rd ed. 1994)) and the references cited therein for a discussion of how to isolate and culture cells from patients).

In one embodiment, stem cells are used in ex vivo procedures for cell transfection and gene therapy. The advantage to using stem cells is that they can be differentiated into other cell types in vitro, e.g., CNS or brain tissue.

Stem cells are isolated for transduction and differentiation using known methods. For example, stem cells are isolated from bone marrow cells by panning the bone marrow cells with antibodies which bind unwanted cells, such as CD4+ and CD8+ (T cells), CD45+ (panB cells), GR-1 (granulocytes), and Iad (differentiated antigen presenting cells) (see Inaba et al., J. Exp. Med. 176:1693-1702 (1992)).

In other embodiments, nucleic acids encoding a ST3Gal-III, can be introduced into CNS cells, brain cells or stem cells ex vivo and then reintroduced into the patient.

Vectors (e.g., retroviruses, adenoviruses, liposomes, etc.) containing therapeutic nucleic acids can also be administered directly to the organism for transduction of cells in vivo. Alternatively, naked DNA can be administered. Administration is by any of the routes normally used for introducing a molecule into ultimate contact with blood or tissue cells. Suitable methods of administering such nucleic acids are available and known to those of skill in the art, and, although more than one route can be used to administer a particular composition, a particular route can often provide a more immediate and more effective reaction than another route.

In some embodiments, transcription of an administered ST3Gal-III nucleic acid sequence is under the control of an inducible or a CNS-specific (brain-specific or neuron specific) promoter. Promoters for inducible expression or tissue-specific expression are known in the art. Exemplified inducible promoters include a metallotheionein promoter or a brain natriuretic peptide promoter. Exemplified brain specific promoters include promoters for actin-binding LIM domain protein (ABLIM) (Klimov, et al., Biochlim Biophys Acta (2005) July 7; PMID 16005990), brain aromatase exon 1f (Harada and Honda, J Steroid Biochem Mol Biol (2005) June 12; PMID 15955692), estrogen receptor alpha and beta (Hamada, et al., Brain Res Mot Brain Res, (2005) June 10; PMID 15953656; and Hu, et al., J Neurobiol (2005) 64:298-309), melanocortin 4 receptor (Daniel, et al., Mol Cell Endocrinol (2005) 239:63-71), and brain natriuretic peptide (Ma, et al., Regul Pept (2005) 128:169-76) genes. Exemplified neuron specific promoters include promoters for the enolase (Pillai-Nair, et al., J Neurosci (2005) 25:4659-71), Thy 1.2 (Araki, et al., Genesis (2005) 42:53-60) and tyrosine hydroxylase (Sorensen, et al., Eur J Neurosci (2005) 21:2793-9) genes.

### Diagnosis of Risk for Disease

In some embodiments, the invention provides methods of diagnosing a risk for an epileptic condition associated with decreased ST3Gal-III activity. Risk for epilepsy can be assessed for example, by determining the presence of one or more mutations in a ST3Gal-III gene that decrease or inhibit the activity of a ST3Gal-III sialyltransferase. Such mutations can lead to decreased levels of Siaα2-3Galβ1-3GlcNAc or a Siaα2-3Galβ1-4GlcNAc oligosaccharide moieties. Methods for detecting ST3Gal-III nucleic acids or mutants thereof are well known. For example, PCR, nucleic acid hybridization methods, and the like can be used to detect a particular mutant.

In other embodiments, disease risk can be assessed, for example, by determining whether mutations are present in ST3Gal-III genes or genes that regulate ST3-Gal-III transcription or translation lead to abnormal expression of ST3Gal-III, particularly in CNS or brain tissues.

### Transgenic Animals

The invention also provides chimeric and transgenic nonhuman animals which contain cells that lack a functional ST3Gal-III sialyltransferase gene. The animals can be used to test therapies of the invention. A “chimeric animal” includes some cells that lack the functional ST3Gal-III gene and other cells that do not have the inactivated gene. A “transgenic animal,” in contrast, is made up of cells that have all incorporated the specific modification which renders the ST3Gal-III gene inactive. While a transgenic animal is capable of transmitting the inactivated sialyltransferase gene to its progeny, the ability of a chimeric animal to transmit the mutation depends upon whether the inactivated gene is present in the animal's germ cells. The modifications that inactivate the gene can include, for example, insertions, deletions, or substitutions of one or more nucleotides. The modifications can interfere with transcription of the gene itself, with translation and/or stability of the resulting mRNA, or can cause the gene to encode an inactive sialyltransferase polypeptide. Mice deficient for ST3Gal-III are described in Ellies, et al, supra.

The claimed methods are useful for producing transgenic and chimeric animals of most vertebrate species. Such species include, but are not limited to, nonhuman mammals, including rodents such as mice and rats, rabbits, ovines such as sheep and goats, porcines such as pigs, and bovines such as cattle and buffalo. Methods of obtaining transgenic animals are described in, for example, Puhler, A., Ed., Genetic Engineering of Animals, VCH Publ., 1993; Murphy and Carter, Eds., Transgenesis Techniques: Principles and Protocols (Methods in Molecular Biology, Vol. 18), 1993; Pinkert, C A, Ed., Transgenic Animal Technology: A Laboratory Handbook, Academic Press, 2003; Houdebine, et al., Animal Transgenesis and Cloning, 2003, John Wiley & Sons.

One method of obtaining a transgenic or chimeric animal having an inactivated ST3Gal-III sialyltransferase gene in its genome is to contact fertilized oocytes with a vector that includes a sialyltransferase-encoding polynucleotide that is modified to contain an inactivating modification. Alternatively, the modified sialyltransferase gene can be introduced into embryonic stem cells (ES). These cells are obtained from preimplantation embryos cultured in vitro. See, e.g., Hooper, M L, Embryonal Stem Cells: Introducing Planned Changes into the Animal Germline (Modern Genetics, v. 1), Int'l. Pub. Distrib., Inc., 1993; Bradley et al. (1984) Nature 309, 255-258. Transformed ES cells are combined with blastocysts from the non-human animal. See, Jaenisch (1988) Science 240: 1468-1474.

### EXAMPLES

The following examples are offered to illustrate, but not to limit the claimed invention.

### Example 1

### ST3Gal-III Deficient Mice

ST3Gal-III deficient mice have been described in Ellies, et al., Blood (2002) 100:3618-3625. Briefly, genomic clones of the ST3Gal-III gene were isolated from a 129/SvJ phage library (Stratagene, La Jolla, Calif.), and Cre-loxP gene targeting constructs prepared by approaches and procedures described in Priatel, et al., Glycobiology (1997) 7:45-56. Mice bearing mutant genotypes were produced and bred by procedures described in Shafi, et al., Proc Natl Acad Sci USA (2000) 97:5735-5739. Genotyping was performed by polymerase chain reaction (PCR) by using oligonucleotide primers:

### LE-110 (5′-CCAGCCAGCAGAGGATCTGATAC) and

LE-115 (5′-CGCAGGGGGCGTTTCTAGAC) to detect the 450-bp ST3Gal-III wild type allele, and LE-110 and rlox (5′-CTCGAATTGATCCCCGGGTAC) to detect the 300-bp ST3Gal-III Δ allele.

### Example 2

### Measurement of Metabolic and Behavioral Parameters

Protocols for the measurement of metabolic and behavioral parameters are described in Angata, et al., J Biol Chem 279:32603-13. Briefly, two separate cohorts of 4-month-old male mice were analyzed. The first consisted of equal numbers of wild-type and ST3Gal-III-deficient littermates. These were assessed in a behavioral test battery modified from that used by McIlwain, et al., Physiol Behav (2001) 73:705-17, and described in Corbo, et al., J Neurosci (2002) 22:7548-57. This included parameters such as metabolic performance, physical appearance, sensorimotor reflexes, motor activity, nociception, acoustic startle, sensorimotor gating, and assessments of learning and memory. Concern that testing mice in such a large battery could influence behavior in any individual task and that multiple assessments increased the probability of a type I statistical error, a second cohort of mice was also analyzed (wild-type, n=16; Δ/Δ, n=15). In the open field test, activity was measured in a 30-min test period in an area of 45×45 cm using a Digiscan apparatus (Accuscan Electronics, Columbus, Ohio). Vertical activity (rearing) and distance (total and center) were recorded.

Passive avoidance analysis involved a two-compartment light/dark apparatus (35×18×30 cm, Coulbourn Instruments, Allentown, Pa.). Each mouse was placed in the lighted compartment. When the animal entered the dark compartment, a guillotine door closed behind and a foot shock of 0.4 mA was delivered through the grid floor of the dark compartment for 3 s. If the mouse did not enter the dark compartment within 10 min, it was excluded from the retrieval test. In the retrieval trial performed 24 h later, the latency for the mice to enter the dark compartment was recorded. The maximum latency was 600 s.

Fear conditioning analyses used chambers (26×22×18 cm high) made of clear Plexiglas placed in a 2×2 array (Med Associates). A video camera was used for recording and analysis (FreezeFrame, Actimetrics, St. Evanston, Ill.). The conditioned stimulus (CS) was an 85-db, 2,800-Hz, 20-s tone, and the unconditioned stimulus was a scrambled foot shock at 0.75 mA presented during the last 3 s of the CS. Mice were placed in the test chamber for 3 min before recording CS and freezing behavior. Freezing was defined as the absence of movement other than breathing, and thresholds were selected via the software of high correlation with human observers. Three CS/unconditioned stimulus pairings were given with 1-min spacing, and freezing during the CS was also recorded. Each mouse was returned to the shock chamber 24 h later, and freezing responses were recorded for 3 min (context test). The chambers were modified to present a different environmental context (shape, odor, color changes), and 2 h later the mice were placed in this novel environment. Freezing behavior was recorded for 3 min before and during three CS presentations (cued conditioning). The time spent freezing was converted to a percent value.

The water maze task constituted a pretraining phase during which all mice from both cohorts were tested for 2 days in a straight-swim pretraining protocol. Mice received 16 trials (8 trials over 2 days) in a 31×60-cm rectangular tank that was located in a different room than the circular tank used in the hidden platform trials. The platform was located 1 cm below the water opposite from the start location. Latency to climb onto the platform was the dependent measure. Criteria for advancing to the hidden platform trials was completing 6 of 8 trials under 10 s on the 2nd day. This pretraining procedure provided experience with swimming and climbing onto a submerged platform without exposing the mice to the spatial cues used in the hidden platform trials. This procedure both screens for mice with severe motor deficits and reduces behavioral variability often seen on the 1st day of hidden platform testing. All mice successfully passed this pretraining phase. Hidden platform testing followed in which extra-maze visual cues were hung from a curtain located around a 1.26-m diameter circular tank. The water was made opaque with the addition of non-toxic paint. The 10-cm diameter escape platform was located 1 cm below the surface of the water, and a Polytrack video-tracking system (San Diego Instruments) was used to collect mouse movement data (location, distance, and latency) during training and probe trials. Each mouse was given eight trials a day, in two blocks of four trials for 4 consecutive days. After 36 trials, each animal was given a 60-s probe trial. During the probe test, the platform was removed, and quadrant search times were measured. Visual cue testing was performed 1 day after the last hidden platform training trial, wherein mice were trained to locate a visible cued platform. The visible cue was a gray plastic cube (9 cm) attached to a pole so that it was 10 cm above the platform. On each trial of the visible platform test, the platform was randomly located in one of the four quadrants. Mice were given eight trials, in blocks of four trials, and the latency to find the platform was recorded for each trial.

Metabolic chambers termed CLAMS (Comprehensive Lab Animal Monitoring System; Columbus Instruments, Columbus, Ohio) automatically recorded metabolic parameters including volume of carbon dioxide produced (VCO2), volume of oxygen consumed (VO2), respiration (respiratory exchange ratio)=VCO2/VO2, and caloric (heat) value ((3.815+1.232×respiratory exchange ratio)×VO2), motion in all three axes in time, and consumption of food and water. Data were collected every 30 min over three 12-h dark cycles and two 12-h light cycles and analyzed as mean values over each 12-h period with the exception of food and water intake which were added to the total during subsequent cycles.

Pulmonary function was scored by measurement of the uptake of CO. A carbon monoxide uptake monitor (Columbus Instruments) measured the CO level in a sealed chamber after exposing the mouse to a 60-s interval of air with 0.17% CO. The mean breath per min was also recorded. Each animal was tested once.

Blood pressure was determined by a noninvasive blood pressure tail-cuff system (Columbus Instruments) that measures systolic blood pressure in addition to heart rate and relative changes in diastolic and mean blood pressure. Individual mice were placed in a small cylinder chamber; occlusion and sensor cuffs were placed on the tail, and the tail was warmed to 37° C. Mice were first acclimated to the restraining chamber, tail cuffs, and the heat fan for 30 min for 2 days prior to testing. The mean of four measurements on the 3rd day was reported and analyzed by the Student's t test.

### Example 3

### Detection of Seizures

Electrocorticographic recordings. Silver wire electrodes (0.005 inch diameter) soldered to a microminiature connector were implanted bilaterally into the subdural space over the frontal and parietal cortex of mice under anesthesia several days before recording. Simultaneous cortical activity and behavioral video/electrographic monitoring was performed using a digital electroencephalograph (Stellate Systems, Montreal, Canada) from ST3GalIII +/+ and Δ/Δ mice moving freely in the test cage for prolonged periods, including sleep (Noebels, et al., (1984) Nature 310:409-411). Seizure behavior was observed directly and annotated on all recordings.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of detecting epilepsy in a mammal, the method comprising detecting ST3Gal-III activity in a sample from the mammal.

2. The method of claim 1, wherein ST3Gal-III activity is detected by detecting the increase in one or more Siaα2-3Galβ1-3/4GlcNAc moieties.

3. The method of claim 1, wherein ST3Gal-III activity is detected by detecting the decrease in one or more Galβ1-3/4GlcNAc moieties.

4. A method of detecting epilepsy in a mammal, the method comprising detecting a carbohydrate structure in a sample from the mammal.

5. The method of claim 4, wherein the carbohydrate structure is one or more Siaα2-3Galβ1-3/4GlcNAc moieties.

6. The method of claim 4, wherein the carbohydrate structure is one or more Galβ1-3/4GlcNAc moieties.

7. A method of identifying an increased risk for an epileptic condition, the method comprising identifying a mutation in a ST3Gal-III gene, wherein the mutation decreases the activity of the encoded ST3Gal-III polypeptide.

8. A method of treating an epileptic condition, the method comprising administering to the mammal a therapeutically effective amount of an agent that increases activity of ST3Gal-III in the mammal.

9. The method according to claim 8, wherein the agent that increases activity of ST3Gal-III in the mammal is a ST3Gal-III polypeptide.

10. The method according to claim 8, wherein the agent that increases activity of ST3Gal-III in the mammal is a nucleic acid encoding ST3Gal-III.

11. The method according to claim 8, wherein the agent that increases activity of ST3Gal-III increases the level of ST3Gal-III protein expression.

12. The method according to claim 8, wherein the agent that increases activity of ST3Gal-III increases the level of ST3Gal-III mRNA expression.

13. A method of increasing the levels of one or more Siaα2-3Galβ1-3/4GlcNAc moieties in a central nervous system (CNS) cell, the method comprising,
introducing into a CNS cell an expression vector comprising a nucleic acid that encodes a ST3Gal-III polypeptide or enzymatically active fragment thereof.

14. The method of claim 13, wherein the expression vector is introduced in the CNS cell in vitro.

15. The method of claim 13, wherein the expression vector is introduced in the CNS cell in vivo.

16. A method of identifying an agent for the treatment of an epileptic condition in an individual, the method comprising identifying a candidate agent that increases the activity of ST3Gal-III.

17. The method of claim 16, wherein the step of identifying the agent comprises contacting the candidate agent with a ST3Gal-III polypeptide or enzymatically active fragment thereof.

18. The method of claim 16, wherein the candidate agent increases the amount of a Siaα2-3Galβ1-3/4GlcNAc in a CNS cell.
